SEQUENCE LISTING (21 sequences)

SEQ ID NO: 1 - protein, 6 aa, synthetic construct
GGGGCC

SEQ ID NO: 2 - DNA, 33 bp, synthetic construct
  source 1..33: mol_type = other DNA
ttgggtctcggaggcagccgagtgtttaagagc

SEQ ID NO: 3 - DNA, 40 bp, synthetic construct
  source 1..40: mol_type = other DNA
ttagctcttaaacactcggctgcctccgagacccaacaag

SEQ ID NO: 4 - DNA, 21 bp, synthetic construct
  source 1..21: mol_type = other DNA
ctgtatcccgtgacatttcat

SEQ ID NO: 5 - DNA, 21 bp, synthetic construct
  source 1..21: mol_type = other DNA
aagcaaagccaagatgagcct

SEQ ID NO: 6 - DNA, 20 bp, synthetic construct
  source 1..20: mol_type = other DNA
aacgaggcctcttctcacaa

SEQ ID NO: 7 - DNA, 20 bp, synthetic construct
  source 1..20: mol_type = other DNA
cctccgtgtagtgacccttg

SEQ ID NO: 8 - DNA, 20 bp, synthetic construct
  source 1..20: mol_type = other DNA
cttacggagcggtcgtgtag

SEQ ID NO: 9 - DNA, 19 bp, synthetic construct
  source 1..19: mol_type = other DNA
agaaggaaacggtgaaggt

SEQ ID NO: 10 - DNA, 19 bp, synthetic construct
  source 1..19: mol_type = other DNA
tcggtgacgacccatccat

SEQ ID NO: 11 - DNA, 23 bp, synthetic construct
  source 1..23: mol_type = other DNA
cgacgtccacttcatttacaaac

SEQ ID NO: 12 - DNA, 22 bp, synthetic construct
  source 1..22: mol_type = other DNA
actaaccacgaccatcaaatcc

SEQ ID NO: 13 - DNA, 21 bp, synthetic construct
  source 1..21: mol_type = other DNA
agtcccacccaacaaagaagg

SEQ ID NO: 14 - DNA, 21 bp, synthetic construct
  source 1..21: mol_type = other DNA
gtcaaggactcggccaactac

SEQ ID NO: 15 - DNA, 21 bp, synthetic construct
  source 1..21: mol_type = other DNA
gatttcaccgtgatctgagca

SEQ ID NO: 16 - DNA, 23 bp, synthetic construct
  source 1..23: mol_type = other DNA
tctacaaccctgaagtgcttgat

SEQ ID NO: 17 - DNA, 21 bp, synthetic construct
  source 1..21: mol_type = other DNA
caatctgcagacagacactgg

SEQ ID NO: 18 - DNA, 20 bp, synthetic construct
  source 1..20: mol_type = other DNA
gagtcaacggatttggtcgt

SEQ ID NO: 19 - DNA, 20 bp, synthetic construct
  source 1..20: mol_type = other DNA
ttgattttggagggatctcg

SEQ ID NO: 20 - DNA, 20 bp, synthetic construct
  source 1..20: mol_type = other DNA
caaggacgctttctacagtg

SEQ ID NO: 21 - DNA, 20 bp, synthetic construct
  source 1..20: mol_type = other DNA
tgaccttaccctcagaactg

(19) United States
(12) Patent Application Publication
(10) Pub. No.: US 2026/0297571 A1
(43) Pub. Date: Oct. 1, 2026

(54) METHODS FOR DECREASING NEURONAL DEATH, INFLAMMATION, AND DEGENERATION

(71) Applicant: The Johns Hopkins University, Baltimore, MD (US)

(72) Inventors: Shuying Sun, Baltimore, MD (US); Zhe Zhang, Baltimore, MD (US)

(21) Appl. No.: 19/481,160

(22) PCT Filed: May 3, 2024
(86) PCT No.: PCT/US2024/027681

§ 371 Date: Nov. 3, 2025

Related U.S. Application Data
(60) Provisional application No. 63/500,148, filed on May 4, 2023.

(51) Int. Cl. C12N 15/11; A61K 31/136; A61K 38/46; A61P 25/28

(52) U.S. Cl. CPC C12N 15/11; A61K 31/136; A61K 38/465; A61P 25/28

(57) ABSTRACT

The present disclosure provides compositions and methods for treating ALS and/or FTD and for reducing neuroinflammation and neurodegeneration, e.g., as caused by dipeptide repeat (DPR) toxicity. Particularly, the disclosure provides methods for improving neuron survival, decreasing neuroinflammation and treating ALS and/or FTD using PTPσ inhibitors.

### CROSS-REFERENCE TO RELATED APPLICATIONS

This application claims the benefit of U.S. Provisional Application No. 63/500,148, filed May 4, 2023, the content of which is herein incorporated by reference in its entirety.

### STATEMENT REGARDING FEDERALLY SPONSORED RESEARCH OR DEVELOPMENT

This invention was made with government support under NS107347, AG072078, and NS113820 awarded by the National Institutes of Health. The government has certain rights in the invention.

### FIELD

The present disclosure provides compositions and methods for treating ALS and/or FTD and for reducing neuroinflammation and neurodegeneration, e.g., as caused by dipeptide repeat (DPR) toxicity. Particularly, the disclosure provides methods for improving neuron survival, decreasing neuroinflammation, and treating ALS and/or FTD using PTPσ inhibitors.

### SEQUENCE LISTING STATEMENT

The content of the electronic sequence listing titled JHU-41625.601.xml (Size: 19,593 bytes; and Date of Creation: May 3, 2024) is herein incorporated by reference in its entirety.

### BACKGROUND

ALS is an adult-onset progressive neurodegenerative disease. The degeneration of upper and lower motor neurons leads to fatal paralysis in about five years from onset. FTD is a neurodegenerative disorder of the frontal and temporal lobes characterized by behavioral and language dysfunction. The two diseases are increasingly recognized to have clinical, pathological, and genetic overlaps. A hexanucleotide repeat expansion in the C9ORF72 gene is the most common genetic cause of both ALS and FTD.

One potential pathogenic mechanism is the aberrant accumulation of dipeptide repeat (DPR) proteins produced by repeat-associated non-AUG (RAN) translation in all six reading frames (poly-GA, poly-GR, poly-PA, poly-PR, and poly-PG) of both sense and antisense RNAs. Abnormal cytoplasmic inclusions of these DPR proteins have been found in cell models and C9ORF72-ALS/FTD (C9) patient post-mortem brain tissues. Toxicities by different DPRs, especially poly-GR/PR, were reported to be more cytotoxic to neurons in multiple in vitro and in vivo models, likely due to nucleolar dysfunction and stress granule impairment resulting from alteration of the liquid-liquid phase separation dynamics of membrane-less organelles. In particular, poly-GR has been shown to correlate with neurodegeneration in C9 patients, implicating the contribution of poly-GR to disease etiology. Understanding and targeting poly-GR mediated toxicity provides insights into disease mechanisms and aids in the development of much needed therapies.

### SUMMARY

Disclosed herein are methods of treating a disorder or disorder comprising administering to a subject in need thereof an effective amount of at least one protein tyrosine phosphatase sigma (PTPσ) inhibitor or a composition thereof. In some embodiments, the disease or disorder is characterized by the accumulation of dipeptide repeat (DPR) proteins. In some embodiments, the disease or disorder is characterized by the accumulation of arginine-rich dipeptide repeats (DPR). In some embodiments, the disease or disorder is a neurodegenerative disease or disorder.

In some embodiments, the disease or disorder is amyotrophic lateral sclerosis (ALS), frontotemporal dementia (FTD), or a combination thereof. In some embodiments, the disease or disorder is C9ORF72-ALS/FTD.

In some embodiments, administration of the at least one PTPσ inhibitor reduces neuronal death, neuroinflammation, and progression of neurodegeneration.

In some embodiments, the PTPσ inhibitor is selected from the group consisting of proteins, nucleic acids, small molecules, and combinations thereof. In some embodiments, the PTPσ inhibitor comprises an antisense oligonucleotide, a small interfering RNA (siRNA), a short hairpin RNA (shRNA), a guide RNA (gRNA) and combinations thereof. In some embodiments, the PTPσ inhibitor comprises Intracellular Sigma Peptide (ISP), DJ001, or a combination thereof.

In some embodiments, the methods further comprise administering at least one additional therapeutic agent.

Also disclosed herein are methods for enhancing survival, decreasing inflammation, and increasing endosome/lysosome function of a target cell comprising contacting a target cell with at least one protein tyrosine phosphatase sigma (PTPG) inhibitor.

In some embodiments, the target cell is a neuron. In some embodiments, the target cell has accumulation of dipeptide repeat (DPR) proteins.

In some embodiments, contacting the target cell comprises providing the PTPσ inhibitor to an organ, tissue, cell, in vitro or ex vivo. In some embodiments, contacting the target cell comprises administering the PTPσ inhibitor to a subject.

In some embodiments, the PTPσ inhibitor is selected from the group consisting of proteins, nucleic acids, small molecules, and combinations thereof. In some embodiments, the PTPσ inhibitor comprises an antisense oligonucleotide, a small interfering RNA (siRNA), a short hairpin RNA (shRNA), a guide RNA (gRNA) and combinations thereof. In some embodiments, the PTPσ inhibitor comprises Intracellular Sigma Peptide (ISP), DJ001, or a combination thereof.

Other aspects and embodiments of the disclosure will be apparent in light of the following detailed description and accompanying figures.

### BRIEF DESCRIPTION OF THE DRAWINGS

FIGS. 1A-1E show CRISPRi screens genetic modifiers of poly-GR mediated toxicity.

FIG. 1A is immunofluorescent staining of FLAG tag detected GFP or GR50 expression after lentivirus infection. FIG. 1B is representative images of GR50 expressing neurons after 1-day and 9-days transduction and the percentage of the surviving neurons quantified and compared to GFP transduced neurons. Each dot represents one individual well. N=10 for GFP expressing i³Neurons, N=9 for GR50 expressing neurons. FIG. 1C is a schematic of a CRISPRi screening paradigm. iPSCs expressing dCas9 were infected with H1 library, followed by differentiating to neurons. GR50 expression was transduced for 9 days. The surviving GFP and GR50 expressing neurons were harvested on day 14. gDNA of neurons were subject to sequencing and analyzed for sgRNA distribution. FIG. 1D is a volcano plot visualizing all genes identified in the screening. Red: Genes induced neuronal death after knocking down. Green: Genes promote GR50 expressing neuron survival after knocking down. Grey: Genes do not make significant difference to neuron survival after knocking down. FIG. 1E is validation of the neuroprotective effect of PTPσ knockdown. The representative image of control and PTPσ knockdown neurons after 9-days GR50 transduction. The percentage of the surviving neurons were quantified and compared. Data are mean±SEM. Two-way ANOVA analysis with Turkey's post hoc analysis. ***, P<0.001, ****, P<0.0001.

FIGS. 2A-2H show PTPσ knockdown restores PI3P level and rescues endolysosomal deficits in poly-GR expressing i³Neurons. FIG. 2A shows a GFP-2×FYVE reporter detects PI3P in i³Neurons. FIG. 2B shows the quantification of GFP fluorescent signals in GR50 expressing i³Neurons and PTPσ knockdown GR50-expressing i³Neurons. Each colored dot represents one cell. Each black dot represents the average fluorescent intensity of one individual well. N=4 wells for each condition. FIG. 2C is the immunofluorescence staining of EEA1 and FIG. 2D is the quantification of EEA1+puncta in GFP and GR50-expressing i³Neurons with or without PTPσ knockdown. N=4 wells for each condition. FIG. 2E is the immunofluorescence staining and FIG. 2F is the quantification of LAMP1 in the GFP and GR50-expressing i³Neurons with or without PTPσ knockdown. N=3 wells for each condition. FIGS. 2G and 2H are the results of a FITC-Dextran pulse chase assay in GR50-expressing i³Neurons and PTPσ knockdown GR50-expressing i³Neurons. Data are mean±sem from 3 biological replica. One-way ANOVA with Turkey's post hoc analysis was performed to compare the difference of averaged data from individual well among groups. *, P<0.05, **, P<0.01, ***, P<0.001, ****, P<0.0001.

FIGS. 3A-3H show PTPσ knockdown promotes PI3P synthesis and endo-lysosomal formation in C9-patient iPSNs. FIG. 3A is a GFP-2×FYVE reporter detecting PI3P in control, C9 iPSNs and its level upon PTPσ knockdown (top). The quantification of GFP puncta in different conditions is shown below. Each colored dot represents one cell. Each black dot represents the average fluorescent intensity of one individual line. FIG. 3B is the immunofluorescence staining and quantification of EEA1 in control, C9 iPSNs and its level upon PTPσ knockdown. FIG. 3C is the immunofluorescence staining and quantification of LAMP1 in control, C9 iPSNs and its level upon PTPσ knockdown. FIG. 3D is a FITC-Dextran pulse chase assay in control, C9 iPSNs and the FITC fluorescence intensity comparisons upon PTPσ knockdown. One-way ANOVA with Turkey's post hoc analysis was performed to compare the difference of averaged data from individual well among groups. FIG. 3E is representative images of patient derived iPSNs after PI staining in the glutamate-induced excitotoxicity assay. The percentage of PI+dead cells were quantified and compared among different treatments. Each dot represents one line. Data are mean±sem. two-way ANOVA with Tukey's post hoc test was used to compare the difference. FIG. 3F is a graph from a competitive ELISA measured PI3P level in non-FTD control (N=9), non-C9-FTD (N=5) and C9-ALS/FTD patient (N=10) postmortem temporal cortex tissue. One-way ANOVA was used to compare the difference. Each dot represents one patient. FIG. 3G is images of EEA1 staining and FIG. 3H is the fluorescence intensity measurement in the cortex of control and C9-ALS/FTD patient postmortem temporal cortex tissue. Each dot represents one patient. Student t test was used to analyze the difference. *, P<0.05, **, P<0.01, ***, P<0.001, ****, P<0.0001.

FIGS. 4A-4J show ISP treatment rescues decreased PI3P synthesis and endolysosomal defects in i³Neurons expressing both poly-GR and poly-PR. FIG. 4A is images of GFP-2×FYVE reporter detecting PI3P in i³Neurons. FIG. 4B is the quantification of GFP fluorescence signal in GR50 expressing i³Neurons and PTPσ knockdown GR50-expressing i³Neurons. Each colored dot represents one cell. Each black dot represents the average fluorescent intensity of one individual well. N=4 wells for each condition. FIG. 4C is the immunofluorescence staining and FIG. 4D is the quantification of EEA1 in GR50-expressing i³Neurons and PTPσ knockdown GR50-expressing i³Neurons. N=4 for each condition. FIG. 4E is the immunofluorescence staining and FIG. 4F is the quantification of LAMP1 in GR50-expressing i³Neurons and PTPσ knockdown GR50-expressing i³Neurons. N=3 for each condition. FIGS. 4G and 4H are the results from a FITC-Dextran pulse chase assay in GR50-expressing i³Neurons and PTPσ knockdown GR50-expressing i³Neurons. Data are mean±sem from 3 biological replica. FIG. 4I is the quantification of surviving cells after ISP treatment in GR50 expressing neurons. Each dot represents one well. FIG. 4J is the quantification of surviving cells after ISP treatment in PR50 expressing neurons. Each dot represents one well. One-way ANOVA with Turkey's post hoc analysis was performed to compare the difference of averaged data from individual well among groups. *, P<0.05, **, P<0.01, ***, P<0.001, ****, P<0.0001.

FIGS. 5A-5F show ISP administration increases PI3P level and promotes endolysosomal formation in C9-patient iPSNs. FIG. 5A shows GFP-2×FYVE reporter detecting PI3P in control, C9 iPSNs and its level upon ISP treatment. The quantification of GFP fluorescence intensity in different conditions. Each colored dot represents one cell. Each black dot represents the average fluorescent intensity of one individual line. FIG. 5B shows EEA1 staining and quantification in control, C9 iPSNs and its level upon ISP treatment. FIG. 5C shows immunofluorescence staining and quantification of LAMP1 in control, C9 iPSNs and its level upon ISP treatment. FIG. 5D shows FITC-Dextran pulse chase assay in control, C9 iPSNs and its level upon ISP treatment. The fluorescence intensity of FITC was analyzed. One-way ANOVA with Turkey's post hoc analysis was performed to compare the difference of averaged data from individual well among groups. FIG. 5E is representative images of C9 iPSNs after PI staining in the glutamate-induced excitotoxicity assay with ISP treatment. The percentage of PI+dead cells were quantified and compared among different treatments. FIG. 5F is representative images of C9 iPSNs after PI staining in the glutamate-induced excitotoxicity assay after DJ001 treatment. About 500 neurons were quantified at each condition. Each dot represents one line. Data are mean±sem. two-way ANOVA with Tukey's post hoc test was used to compare the difference. ANOVA with Turkey's post hoc analysis. *, P<0.05, **, P<0.01, ****, P<0.0001.

FIGS. 6A-6H show ISP administration ameliorates neurodegeneration and behavioral defects in 3-month old GR100 mice. FIG. 6A is a schematic of ISP treatment of GR100 mice. FIG. 6B is results from an ELISA assay performed to measure PI3P level of PBS or ISP treated mice brains. N=7 for each treatment. FIG. 6C is immunofluorescent staining of EEA1 and quantitative analyses of the EEA1⁺ signal density in the cortex of GFP and GR100 mice. N=12 for PBS control, N=9 for ISP treatment group. FIG. 6D is immunofluorescent staining of NeuN and the quantitative analysis of the NeuN⁺ cell density in the cortex of GFP and GR100 mice. N=4 for PBS, N=5 for ISP group. FIG. 6E is immunofluorescent staining and the quantitative analysis of GFAP and Iba1 in the cortex of GFP and GR100 mice. N=4 for PBS, N=5 for ISP. FIG. 6F is a rotarod test performed to test the motor function of mice. N=20 for PBS, N=17 for ISP. FIG. 6G is results of an open field test used to evaluate the general movement and anxiety level of the mice. Total grid break and rearing represent the horizonal and vertical movement separately. N=20 for PBS, N=17 for ISP. FIG. 6H is results of a novel object recognition test used to probe the learning and memory function of the mice. The percentage of time mice exploring a novel object reflects their ability to distinguish a familiar and a novel object. N=20 for PBS, N=16 for ISP. Each dot presents one mouse. Data is represented as meant sem. Student's t-test was used to calculate statistical significance, *, P<0.05; **, P<0.01; ***, P<0.001.

FIGS. 7A-7I show GR50 transduction induces neurotoxicity in i³Neurons. FIG. 7A shows iPSC containing mNGN2 was differentiated towards neuron in N2 which is supplied with DOX and further differentiated into glutamatergic cortical neuron after 14 days culture in BP. FIG. 7B shows cell morphology of different stages. Scale bar represents 100 μm. FIG. 7C shows neuronal markers MAP2, Tuj1 and NeuN are highly expressed in iPSNs. FIG. 7D shows the mRNA expression levels of multiple neuronal markers were dramatically increased in day 14 i3N compared to the levels in iPSC. FIG. 7E shows day 30 i³Neurons exhibit spontaneous firing. FIG. 7F shows efficient reduction of FUS in the sgRNA-expressing iPSCs and differentiated neurons compared to the non-targeting control. FIG. 7G shows quantitative analyses of neural survival after GFP/GR50 transduction. Data are mean±sem from three biological replicates. Two-way ANOVA with Sidak's multiple comparison test was used to calculate statistical significance. ***, P<0.001, ****, P<0.0001. FIG. 7H shows Propidium iodide (PI) staining and quantification of the dead neurons after 9-days GR50 transduction. Data are indicated as mean #sem. Each dot represents one well. N=10 for each group. Unpaired Student's t-test was used to calculate statistical significance, **** p<0.0001. FIG. 7I shows Western blot and immunofluorescent staining of cleaved caspase 3 (CC3) in GR50 expressing neurons.

FIGS. 8A-8E show PTPσ knockdown does not rescue poly-GA and TDP-43 knockdown mediated neurotoxicity. FIG. 8A shows PTPσ mRNA level in control and PTPσ knockdown i³Neurons. N=4 for each group. Data are indicated as mean±sem. Each dot represents one well. Unpaired Student's t-test was used to calculate statistical significance, ***, p<0.001. FIG. 8B shows PI staining and quantification of the dead neurons in GFP or GR50 expressing control and PTPσ knockdown i³Neurons. Data are indicated as mean±sem. Each dot represents one well. N=3 for control i³Neurons, N=6 for PTPσ knockdown i³Neurons. One-way ANOVA was performed to compare the difference among different treatments followed by Tukey's multiple comparisons test. ****, P<0.0001. FIG. 8C shows representative images and quantification of 9-days GA50 transduced control and PTPσ knockdown i³Neurons. N=6 for control i³Neurons, N=8 for PTPσ knockdown i³Neurons. FIG. 8D shows a diagram of TDP-43 shRNA lentiviral transduction, top, and representative images and quantification of 7-days TDP-43 knockdown control and PTPσ knockdown i³Neurons, bottom. Data are indicated as mean±sem. N=4 for control i³Neurons, N=8 for PTPσ knockdown i³Neurons. Two-way ANOVA with Sidak's multiple comparison test was used to calculate statistical significance. ****, P<0.0001. FIG. 8E shows immunofluorescent staining and western blot of poly-GR-FLAG in control and PTPσ knockdown i³Neurons after 3-days GR50 transduction.

FIGS. 9A-9I show that manipulating PI3P level influences the endolysosome formation. FIG. 9A is images showing the alteration of GFP-2×FYVE fluorescent signal after PTPσ knockdown in U2OS cells. FIG. 9B shows the toxicity of VPS34 inhibitor, VPS34-IN1. FIG. 9C is images of i³Neurons response to VPS34-IN1treatment. FIG. 9D shows neuron survival after VPS34-IN1treatment in BFP or GR50 expressing control and PTPσ knockdown i³Neurons. Data are indicated as mean±sem. N=2 for each condition. FIG. 9E shows the toxicity of PIKFYVE inhibitor, YM-201636. FIG. 9F shows images of the i³Neurons response to YM-201636 treatment in a dose-dependent manner. FIG. 9G shows the quantification of EEA1 size and fluorescence intensity after YM-201636 treatment of different dose. N=3 for each condition. FIG. 9H shows the quantification of surviving neurons after 0.5 μM YM-201636 treatment. Each dot represents one well. N=4 for each group. Data is represented as meant sem. One-way ANOVA was performed to compare the difference among different treatments followed by Tukey's multiple comparisons test. ****, P<0.0001. FIG. 9I is a diagram of PI3P production and its regulatory role in autophagy and early endosome formation.

FIGS. 10A and 10B show EEA1⁺ early endosome reduction is observed GR50-expressing neurons, and absent from GA50 expressing neurons and TDP-43 knockdown neurons. FIG. 10A is immunofluorescent staining and quantitative analyses of EEA1 signals in the 3-days BFP, GA50 and GR50 expressing neurons. FIG. 10B is immunofluorescent staining and quantitative analyses of EEA1 signals in control and TDP-43 knockdown i³Neurons (7 days). Each dot represents one well. Data is represented as meant sem. Student's t test or one-way ANOVA was performed to compare the difference among different treatments followed by Tukey's multiple comparisons test. *, P<0.05; ***, P<0.001.

FIGS. 11A-11E show overexpressing Rab5 (Q79L), but not ATG14L improves the survival of GR50 expressing neurons. FIG. 11A shows Rab5 is overexpressed in the i³Neuron after lentivirus transduction. FIG. 11B shows neuron survival after Rab5 (Q79L) overexpression in BFP or GR50 expressing control and PTPσ knockdown i³Neurons. Data are indicated as mean±sem. N=3 for each condition. FIG. 11C shows ATG14L mRNA level after ATG14L lentivirus transduction. FIG. 11D shows neuron survival after ATG14L overexpression in BFP or GR50 expressing control and PTPσ knockdown i³Neurons. FIG. 11E is representative images and the quantification of EEA1⁺ puncta in BFP and GR50 i³Neurons overexpressing ATG14L or Rab5A (Q79L). Scale bar, 5 μm. n=3 biological replicates for each group. Data are indicated as mean±sem. N=6 for each condition. One-way ANOVA was performed to compare the difference among different treatments followed by Tukey's multiple comparisons test. ***, P<0.001; ****, P<0.0001.

FIGS. 12A-12D show the validation of PTPσ in patient iPSNs. FIG. 12A is a diagram of iPSN differentiation. FIG. 12B is immunofluorescence image of MAP2 in day-32 differentiated neurons. FIG. 12C is Western blotting of PTPσ showed knockdown efficiency in iPSNs. FIG. 12D shows PI3P level in the motor cortex of non-FTD control and C9-ASL/FTD patient postmortem brain tissues. Data is represented as meant sem. Student's t-test was used to calculate statistical significance.

FIGS. 13A-13D show PTPσ inhibitor, DJ001, improves GR50- and PR50-expressing i³Neuorn survival. FIG. 13A shows immunofluorescent staining of EEA1 and quantitative analyses of EEA1⁺ puncta in the DMSO or 3.7 μM DJ001 treated 3-days BFP or GR50 transduced i³Neurons. N=3 individual wells for each treatment. Scale bar represents 5 μm. FIG. 13B shows representative image and quantitative analyses of the survival of 9-days GR50-expressing i³Neuron with and without 3.7 μM DJ001 treatment. Scale bar represents 50 μm. Each dot represents one well. N=6 for each group. Data is represented as meant sem. FIG. 13C is a graph of GR50-expressing neuron survival after DJ001 treatment. Data are indicated as mean±SEM from 6 biological replicates for each treatment. FIG. 13D is a graph of PR50 expressing neuron survival after DJ001 treatment. Data are indicated as mean±SEM from 4 biological replicates. One-way ANOVA was performed to compare the difference among different treatments followed by Tukey's multiple comparisons test. ***, P<0.001; ****, P<0.0001.

FIGS. 14A-14K show 1.5-month old GR100 mice exhibit neurodegeneration. FIG. 14A is a schematic of GR100 construct. FIG. 14B is immunofluorescent staining of poly-GR in the cortex of mouse brain. FIG. 14C is a graph of the brain weight of GFP and GR100 mice. N=9 for each group. FIG. 14D is the immunofluorescent staining of NeuN in the cortex of GFP and GR100 mice. FIG. 14E is the quantitative analysis of the NeuN⁺ cell density in the cortex of GFP and GR100 mice. N=3 for each group. FIG. 14F is the immunofluorescent staining of GFAP and Iba1 in the cortex of GFP and GR100 mice. FIG. 14G is the quantitative analysis of the GFAP⁺ cell density in the cortex of GFP and GR100 mice. FIG. 14H is the quantitative analysis of the Iba1⁺ cell density in the cortex of GFP and GR100 mice. N=4 for GFP control, N=6 for GR100 mice. FIG. 14I is results from a rotarod test performed to test the motor function of mice. The latency to fall(s) reflects movement and coordination ability. N=9 for GFP control, N=10 for GR100 mice. FIG. 14J is immunofluorescent staining of EEA1 and quantitative analyses of the EEA1+signal density in the cortex of GFP and GR100 mice. N=4 for GFP control, N=5 for GR100 mice. FIG. 14K is a PI3P ELISA assay performed to measure PI3P level of GFP and GR100 mouse cortex. N=6 for each group. Data is represented as meant sem. Student's t-test was used to calculate statistical significance, *, P<0.05; **, P<0.01.

FIGS. 15A-15L show 3-month old GR100 mice exhibit progressive neurodegeneration and behavioral defects. FIG. 15A is a graph of the body weight of GFP and GR100 mice at 3 months. N=16 for GFP control, N=17 for GR100 mice. FIG. 15B shows the brain weight of GFP and GR100 mice. N=8 for GFP control, N=6 for GR100 mice. FIG. 15C shows immunofluorescent staining of NeuN in the cortex of GFP and GR100 mice. FIG. 15D shows the quantitative analysis of the NeuN⁺ cell density in the cortex of GFP and GR100 mice. N=6 for each group. FIG. 15E shows the immunofluorescent staining of GFAP and Iba1 in the cortex of GFP and GR100 mice. FIG. 15F shows the quantitative analysis of the GFAP⁺ cell density in the cortex of GFP and GR100 mice. N=6 for each group. FIG. 15G shows the quantitative analysis of the Iba1⁺ cell density in the cortex of GFP and GR100 mice. N=6 for each group. FIG. 15H shows the results from a rotarod test performed to test the motor function of mice. N=17 for GFP control, N=18 for GR100 mice. FIG. 15I shows an open field test was used to evaluate the general movement and anxiety level of the mice. Total grid break and rearing represent the horizonal and vertical movement separately. N=17 for GFP control, N=18 for GR100 mice. FIG. 15J shows results from a novel object recognition test used to probe the learning and memory function of the mice. The percentage of time mice exploring a novel object reflects their ability to distinguish a familiar and a novel object. N=9 for each group. FIG. 15K shows immunofluorescent staining of EEA1 and quantitative analyses of the EEA1+signal density in the cortex of GFP and GR100 mice. N=4 for GFP control, N=5 for GR100 mice. FIG. 15L is ELISA measurements of PI3P levels in mouse brain (n=7). Each dot represents one animal. Data is represented as meant sem. Student's t-test was used to calculate statistical significance, *, P<0.05; **, P<0.01; ***, P<0.001; ****, P<0.0001.

FIGS. 16A and 16B show ISP administration increases body weight and brain weight in GR100 mice. FIG. 16A is a graph of the body weight of GR100-PBS (N=16) and GR100-ISP mice (N=17) after 1.5 months ISP treatment. FIG. 16B shows the gross morphology of PBS (left) and ISP (right) treated GR100 mouse brain. The brain weight of PBS (N=20) and ISP (N=18) treated GR100 mouse brains. Data is represented as meant sem. Student's t-test was used to calculate statistical significance, *, P<0.05.

FIGS. 17A-17E show PTPσ is a strong modifier of poly-PR mediated neurotoxicity. FIG. 17A is a graph of the survival of neurons expressing different inducible DPRs. Each dot represents one biological replicate. Neuron survival are normalized to iGFP CTRL group and indicated as mean±SEM from 3-5 biological replicates. ns, no significant difference, *P=0.0438, ***P=0.0001, ****P<0.0001, by one-way ANOVA with Turkey's post hoc analysis. FIG. 17B is a graph of neuron survival of PR50 expressing neurons after VPS34 IN1 treatment. n=4 biological replicates for each treatment. Data are mean±SEM. Two-way ANOVA was performed to compare the differences among treatments followed by Sidak's multiple comparison test. ****P<0.0001. FIG. 17C is representative images and the normalized fluorescent intensity of GFP-2×FYVE reporter in PR50 expressing i³Neurons. Scale bar, 5 μm. Each symbol represents one cell and different shapes represent different biological replicates. The black symbols show the mean of each replicate, which was used for the statistical analysis. n=6 biological replicates for CTRL shRNA BFP; n=6 replicates for CTRL shRNA PR50; n=6 replicates for PTPσ shRNA BFP PTPσ; n=5 replicates for shRNA PR50. Data are mean±SEM. ****P<0.0001, by one-way ANOVA with Turkey's post hoc analysis. FIG. 17D is representative images and the quantification of EEA1+puncta in GFP and PR50 expressing i³Neurons with or without PTPσ knockdown. Scale bar, 5 μm. Data are mean±SEM from 3 biological. * P=0.0164 (left) and *P=0.0158 (right), respectively, by one-way ANOVA with Turkey's post hoc analysis. FIG. 17E is representative images and quantification of LAMP1 in PR50 expressing neurons with CTRL or PTPσ sgRNA. Scale bar, 5 μm. Data are mean±SEM from 3 biological replicates. ****P<0.0001, by one-way ANOVA with Turkey's post hoc analysis.

FIGS. 18A-18D show that ASOs targeting PTPσ improve neuron survival in poly-GR/PR expressing neurons and C9-iMNs. FIG. 18A is western blotting for PTPσ expression in i³Neurons after ASO treatment. FIG. 18B is a timeline of ASO treatment in R-DPR expressing neurons and C9-iMNs. FIG. 18C is a graph of the survival of GR50/PR50 expressing neurons after 7-day PTPσ ASO treatment. The data was normalized to the averaged survival rate of BFP CTRL ASO group. Each dot represents one biological replicate. n=4 for BFP, n=6 for GR50 and PR50. Data are mean±SEM. ****P<0.0001, by two-way ANOVA with Tukey's post hoc test. FIG. 18D is a graph of the glutamate-induced excitotoxicity assay in C9-ALS/FTD patient lines after PTPσ ASO treatment. Each dot represents one iPSC line. n=3 for control lines; n=4 for C9 lines. Data are mean±SEM. ****P<0.0001, by two-way ANOVA with Tukey's post hoc test.

### DETAILED DESCRIPTION

The present disclosure provides compositions and methods for treating ALS and/or FTD and for reducing neuroinflammation and neurodegeneration, e.g., as caused by dipeptide repeat (DPR) toxicity.

As disclosed herein, two screening strategies were adapted to identify modifiers of dipeptide repeat (DPR) toxicity including poly-GR toxicity. A CRISPRi-i³Neuron based platform was adapted to identify the druggable modifiers that can improve poly-GR expressing neuron survival. Using this system coupled with a druggable CRISPRi sub library, PTPσ was identified as a key regulator of poly-GR mediated neurotoxicity. Knocking down PTPσ or PTPσ inhibition improved neuron survival and restored homeostasis of endo-lysosome in poly-GR expressing neurons and C9-iPSNs. Treating GR100 mice with ISP, a PTPσ inhibitor rescued the behavioral phenotype and ameliorate cellular phenotypes.

PTPσ inhibitor, ISP, is a good candidate for future translational studies. The TAT sequence enables the drug to penetrate BBB. Chronic administration of ISP for up to 3 months is safe. Herein, ISP showed comparable rescue effect in all the in vitro assays as PTPσ knockdown. More excitingly, in the vivo experiment, ISP treatment was started in GR100 mice 1.5 months after GR100 expression when significant neuronal loss and neuroinflammation already present. One and a half months of ISP administration successfully stopped the progression of neurodegeneration, reflected by improved behavior and cellular phenotypes. ISP treatment after the disease onset may improve neuronal survival and function in C9 patients.

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

“Antibody” and “antibodies” as used herein refers to monoclonal antibodies, monospecific antibodies (e.g., which can either be monoclonal, or may also be produced by other means than producing them from a common germ cell), multi-specific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25 (11): 1290-1297 (2007) and PCT International Application WO 2001/058956, the contents of each of which are herein incorporated by reference), or domain antibodies (dAbs) (e.g., such as described in Holt et al., Trends in Biotechnology 21:484-490 (2014)), and including single domain antibodies sdAbs that are naturally occurring, e.g., as in cartilaginous fishes and camelid, or which are synthetic, e.g., nanobodies, VHH, or other domain structure), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass. For simplicity sake, an antibody against an analyte is frequently referred to herein as being either an “anti-analyte antibody” or merely an “analyte antibody.”

“Antibody fragment” as used herein refers to a portion of an intact antibody comprising the antigen-binding site or variable region. The portion does not include the constant heavy chain domains (i.e., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

The term “contacting” as used herein refers to bring or put in contact, to be in or come into contact. The term “contact” as used herein refers to a state or condition of touching or of immediate or local proximity. Contacting inhibitors of the disclosed methods to a target destination, such as, but not limited to, an organ, tissue, cell, or tumor, may occur by any means of administration known to the skilled artisan.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The proteins may be modified by the addition of sugars, lipids or other moieties not included in the amino acid chain. The terms “polypeptide” and “protein” are used interchangeably herein.

“Polynucleotide” or “oligonucleotide” or “nucleic acid,” as used herein, means at least two nucleotides covalently linked together. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. Polynucleotides may be single- or double-stranded or may contain portions of both double stranded and single stranded sequence. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

As used herein, the terms “providing,” “administering,” and “introducing,” are used interchangeably herein and refer to the placement of the compositions of the disclosure into a subject by a method or route which results in at least partial localization to a desired site. The compositions can be administered by any appropriate route which results in delivery to a desired location in the subject.

As used herein, “treat,” “treating,” and the like means a slowing, stopping, or reversing of progression of an infection, disease, disorder and/or condition when provided a composition described herein to an appropriate subject. The term also includes a reversing of the progression of such an infection, disease, disorder and/or condition to a point of eliminating or greatly reducing the disease. As such, “treating” means an application the compositions described herein to a subject, where the subject has a disease, disorder and/or condition or a symptom of a disease, disorder and/or condition, where the purpose is to cure, heal, alleviate, relieve, alter, remedy, ameliorate, improve, or affect the disease, disorder and/or condition or symptoms of the disease, disorder and/or condition.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Protein Tyrosine Phosphatase Sigma (PTP6) Inhibitors

Tyrosine phosphorylation is a main mechanism for regulating protein functions and signal transduction in multiple cell types. Protein tyrosine phosphatases (PTPs) are a large family of enzymes that remove phosphate groups and result in changing functionality and modulating signal transductions. Receptor-type PTPs (RPTPs) exhibit a distinct spatial pattern of expression. PTPσ, a RPTP, is expressed in various immune cells and regulates differentiation, production of various cytokines and immune responses. PTPσ is highly expressed throughout neural development and remains important for neural plasticity even in the adult brain.

PTPσ inhibitors include any substance (e.g., nucleic acid, proteins, polysaccharides, nucleotides, amino acids, monosaccharides or simple sugars, small molecules) which inhibits the transcription, translation, or function of PTPσ. In some embodiments, the inhibitor is specific for PTPσ over other PTPs.

In some embodiments, PTPσ inhibitors block activation of or binding of an agonistic or antagonistic ligand to PTPσ, inhibit the PTPσ induced signaling cascade, or inhibit phosphatase activity. In some embodiments, PTPσ inhibitors disrupt the transcription or translation of PTPσ. Suitable PTPσ inhibitors include, but are not limited to, gene silencing oligonucleotides (e.g., an siRNA, an antisense oligonucleotide, dominant-negative, a short-hairpin RNA, a miRNA, a dicer-substrate RNA, a DNAzyme, or an aptamer targeting the PTPσ gene or the PTPσ messenger RNA), an anti-PTPσ antibody (e.g., a monoclonal, polyclonal, murine, chimeric, humanized, or human antibody targeting an PTPσ epitope or PTPσ ligand, thus interfering with PTPσ activity or ligand binding), a small molecule inhibitor of PTPσ, a negative allosteric modulating agent, a dominant negative PTPσ, a fragment of PTPσ or a related PTP, a CRISPR knockout systems or a combination thereof, or nucleic acid encoding thereof, as appropriate. In some embodiments, the inhibitors include substances and systems which modulate the action of the gene product, including but not limited to, antibodies and small molecule inhibitors.

In some embodiments, the PTPσ inhibitor is Intracellular Sigma Peptide (ISP). ISP is a cell-permeable, TAT conjugated peptide derived from the PTPσ wedge domain that binds to PTPσ and relieves glial-derived chondroitin sulfate proteoglycan (CSPG)-mediated inhibition. NVG-291 is a therapeutic peptide derived from the intracellular domain of PTPσ, similar to ISP, which is developed independently by NervGen. In some embodiments, the PTPσ inhibitor is DJ001 ((Z)-3-((3-nitrophenyl)amino)-1-phenylprop-2-en-1-one), or a stereoisomer, enantiomer, or analog thereof.

### 3. Methods

The disclosure provides methods of treating or preventing a disease or disorder in a subject comprising administering to the subject an effective amount of at least one protein tyrosine phosphatase sigma (PTPG) inhibitor, or a composition thereof. In some embodiments, disease or disorder is characterized by the accumulation of dipeptide repeat (DPR) proteins. In some embodiments, disease or disorder is neurodegenerative disease or disorder.

In some embodiments, the disease or disorder is amyotrophic lateral sclerosis (ALS), frontotemporal dementia (FTD), or a combination thereof. ALS and FTD are severe neurodegenerative diseases with no effective treatment. Degeneration of the upper and lower motor neurons in ALS leads to progressive paralysis. Depending on the affected regions, FTD patients suffer from dementia, behavioral abnormalities, language impairment and personality changes. Both FTD and ALS are regarded as spectrum disorders and their clinical, pathological, and genetic characteristics overlap substantially, and are often described as extreme ends of a disease spectrum. The most common genetic cause of both diseases is a GGGGCC (G₄C₂) (SEQ ID NO: 1) repeat expansion either in the first intron or the promoter region of C9ORF72. Effected individuals typically have several hundred or thousand repeats, whereas healthy controls show less than 33 repeats. Despite the lack of an ATG start codon, RNAs transcribed from these repeats in both sense and antisense directions can be translated into five dipeptide repeat (DPR) proteins-poly (GA), poly (GR), poly (GP), poly (PR), and poly(PA), which form insoluble, ubiquitinated, p62-positive aggregates that are most abundant in the cerebral cortex and cerebellum. Among them, poly (GR) is believed to be a key neurotoxic species, as its expression strongly correlates with neurodegeneration in the brains of patients with C9ORF72 mutations. Moreover, poly (GR) is toxic in many cellular and animal models. In select embodiments, the disease or disorder is C9ORF72-ALS/FTD.

When utilized as a method of treatment, the effective amount may depend on the particular inhibitor, the individual patient parameters including age, physical condition, size, gender and weight, the duration of the treatment, the nature of concurrent therapy (if any), the specific route of administration and like factors within the knowledge and expertise of the health practitioner.

The described inhibitors may be utilized as a composition which further comprises an excipient or pharmaceutically acceptable carrier. The choice of excipients or pharmaceutically acceptable carriers will depend on factors including, but not limited to, the particular mode of administration, the effect of the excipient on solubility and stability, and the nature of the dosage form.

Excipients and carriers may include any and all solvents, dispersion media, antibacterial and antifungal agents, isotonic and absorption delaying agents. Some examples of materials which can serve as excipients and/or carriers are sugars including, but not limited to, lactose, glucose and sucrose; starches including, but not limited to, corn starch and potato starch; cellulose and its derivatives including, but not limited to, sodium carboxymethyl cellulose, ethyl cellulose and cellulose acetate; powdered tragacanth; malt; gelatin; talc; excipients including, but not limited to, cocoa butter and suppository waxes; oils including, but not limited to, peanut oil, cottonseed oil, safflower oil, sesame oil, olive oil, corn oil and soybean oil; glycols; including propylene glycol; esters including, but not limited to, ethyl oleate and ethyl laurate; agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as coloring agents, releasing agents, preservatives, and antioxidants. Techniques and formulations may be found, for example, in Remington's Pharmaceutical Sciences, 19th Edition (Mack Publishing Company, 1995).

The phrase “pharmaceutically acceptable,” as used in connection with compositions and/or cells of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants.

The compositions may be formulated for any appropriate manner of administration, and thus administered, including for example, oral, nasal, intraocular, intravenous, intravaginal, epicutaneous, sublingual, intracranial, intradermal, intraperitoneal, subcutaneous, intramuscular administration, or via inhalation. Techniques and formulations may generally be found in “Remington's Pharmaceutical Sciences,” (Meade Publishing Co., Easton, Pa.). Therapeutic or pharmaceutical compositions must typically be sterile and stable under the conditions of manufacture and storage.

In some embodiments, the described inhibitors, or compositions thereof, are delivered by, for example, an intramuscular, intravenous, transdermal, intranasal, oral, mucosal, or other delivery method.

The amount of the PTPσ inhibitor or a composition thereof required for use in the disclosed methods will vary not only with the particular PTPσ inhibitor or composition selected but also with the route of administration, the nature and/or symptoms of the disease and the age and condition of the patient and will be ultimately at the discretion of the attendant physician or clinician. The determination of effective dosage levels, that is the dosage levels necessary to achieve the desired result, can be accomplished by one skilled in the art using routine methods, for example, human clinical trials, in vivo studies, and in vitro studies. For example, useful dosages of a PTPσ inhibitor, or composition thereof, can be determined by comparing their in vitro activity, and in vivo activity in animal models.

Dosage amount and interval may be adjusted individually to provide plasma levels of the active agent which are sufficient to maintain the modulating effects, or minimal effective concentration (MEC). The MEC will vary for each PTPσ inhibitor but can be estimated from in vivo and/or in vitro data. Dosages necessary to achieve the MEC will depend on individual characteristics and route of administration. However, bioassays can be used to determine plasma concentrations. Dosage intervals can also be determined using MEC value. PTPσ inhibitors or a composition thereof should be administered using a regimen, which maintains plasma levels above the MEC for 10-90% of the time, preferably between 30-90% and most preferably between 50-90%. In cases of local administration or selective uptake, the effective local concentration of the PTPσ inhibitor may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate, precluding toxicity. The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be also used in veterinary medicine for non-human subjects.

PTPσ inhibitors or a composition thereof can be evaluated for efficacy and toxicity using known methods. For example, the toxicology of a particular PTPσ inhibitor may be established by determining in vitro toxicity towards a cell line, such as a mammalian, and preferably human, cell line. The results of such studies are often predictive of toxicity in animals, such as mammals, or more specifically, humans. Alternatively, the toxicity of a particular PTPσ inhibitor in an animal model, such as mice, rats, rabbits, dogs, or monkeys, may be determined using known methods. The efficacy of a particular PTPσ inhibitor may be established using several recognized methods, such as in vitro methods, animal models, or human clinical trials. When selecting a model to determine efficacy, the skilled artisan can be guided by the state of the art to choose an appropriate model, dose, route of administration and/or regime.

A wide range of second therapies may be used with the disclosed methods. The second therapy may be administration of a therapeutic agent or may be a second therapy not connected to administration of another agent (e.g., breathing care, mechanical ventilation, physical therapy, occupational therapy, speech therapy, nutritional support). In some embodiments, the second therapy may be a therapeutic agent (e.g., benzothiazoles (e.g., riluzole), antioxidants (e.g., edaravone), sodium phenylbutyrate/taurursodiol, antidepressants (e.g., serotonin uptake inhibitors (SSRIs)), antipsychotics).

The second therapy may be administered at the same time as the disclosed methods, either in the same composition or in a separate composition administered at substantially the same time. In some embodiments, the second therapy may precede or follow the disclosed methods by time intervals ranging from hours to months.

The disclosure provides methods of enhancing survival, decreasing inflammation, and increasing endosome/lysosome function of a target cell. In some embodiments, the methods comprise contacting a target cell with a PTPσ inhibitor.

The methods are not limited by the type or nature of the target cell. The target cell may be any cell type. In some embodiments, the target cell is a neuron. In some embodiments, the target cell has accumulation of dipeptide repeat (DPR) proteins.

The target cell may be a cell in vitro, either from a cell line or cells obtained from a subject (ex vivo). In some embodiments, contacting the target cell comprises providing the PTPσ inhibitor to an organ, tissue, cell, in vitro or ex vivo.

The target cell may be in vivo. Thus, in some embodiments, contacting the target cell comprises administering the PTPσ inhibitor to a subject.

### 4. EXAMPLES

### Example 1

### CRISPRi-Cas9 Screens for Modifiers of Poly-GR Mediated Toxicity in i³Neuorns

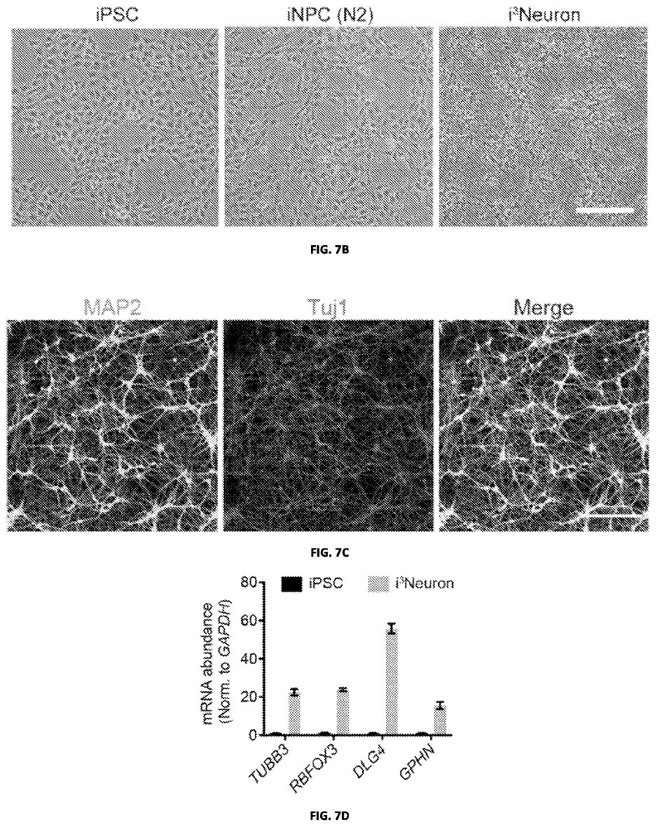

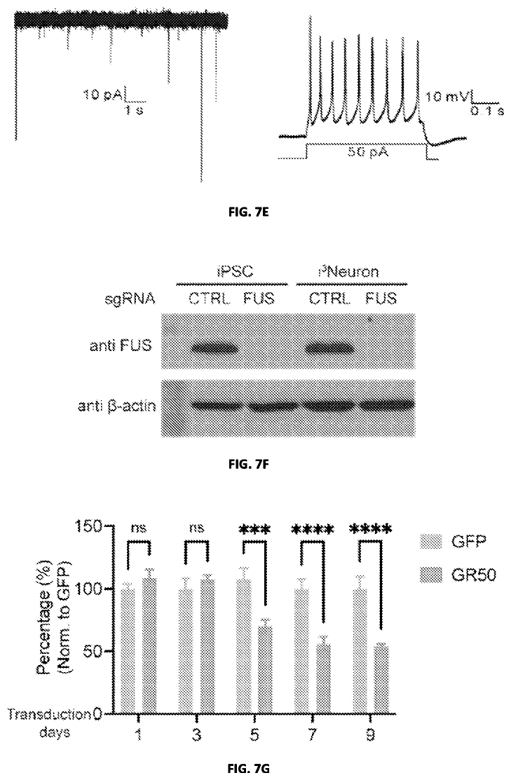

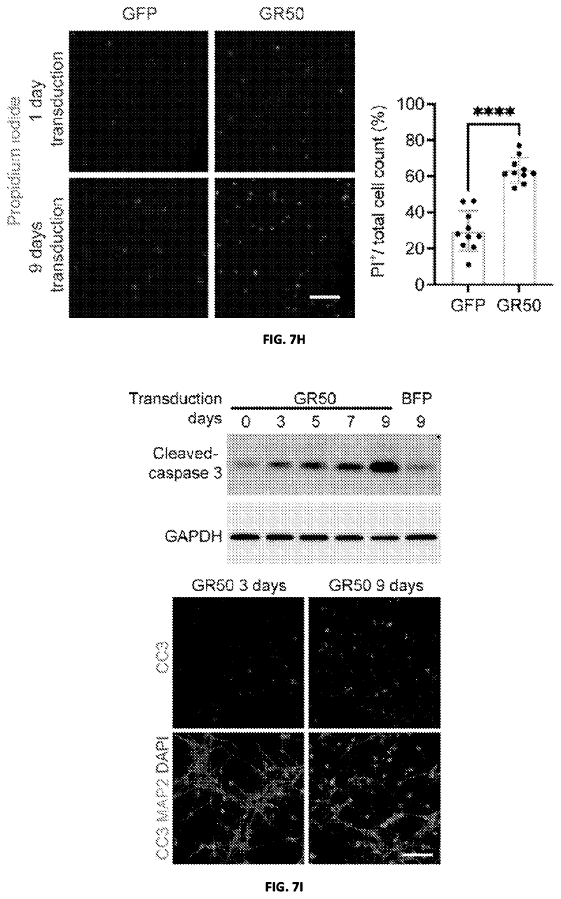

To identify novel genetic modifiers that can reduce poly-GR mediated toxicity in human neurons, a CRISPRi-i³Neuron based screening platform (Tian, R., et al. Neuron 104, 239-255.e212 (2019)) was adapted. Briefly, the engineered human iPSC contains doxycycline-inducible neurogenin (NGN2) in the AAVS1 safe harbor locus. Upon induction with doxycycline, the cells will start differentiating and yield large quantities of highly homogenous cortical neurons (i³Neuron) within 14 days (FIGS. 7A-7D). In addition, an expression cassette of CAG promoter-driven dCas9-BFP-KRAB was engineered into the CLYBL safe harbor locus, which can repress the targeted gene transcription through specific sgRNAs in i³Neuons (FIG. 7E).

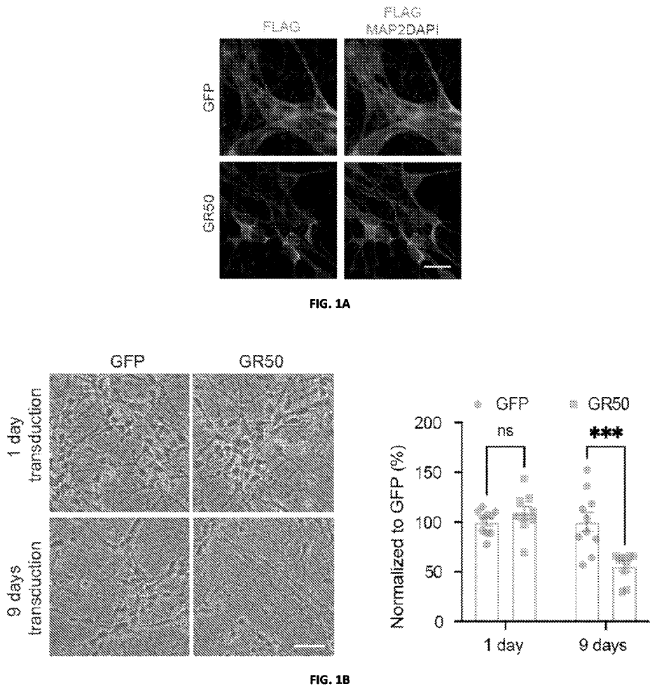

Either 50 repeats of GR dipeptide (GR50) or GFP control tagged by N-terminus APEX2 and C-terminus FLAG as a negative control was expressed in day-5 i³Neurons via lentiviral transduction and harvested on day 14. Immunofluorescence (IF) staining showed that GR50 was predominantly expressed in the soma of the neurons (FIG. 1A). Significantly increased cell death of neurons 9 days after poly-GR transduction was found following counting of the surviving neurons (FIGS. 1B and 7F). This finding was further confirmed by propidium iodide (PI) staining of dead cells, suggesting poly-GR is toxic in the human i³Neuron (FIGS. 7G and 7H). The expression of cleaved-caspase 3 (CC3), an apoptosis marker, from 3 to 9 days post GR50 transduction was also measured. A mild elevation of CC3 was evident after 3 days, which became more pronounced with prolonged transduction (FIG. 7I). These observations confirm that GR50 induces time-dependent toxicity in i³Neurons. As such, this provided a suitable platform for survival-based screening used throughout.

To increase the chance of translating the result of the hits from the current screening to therapeutic development, the screening was performed using the CRISPRi v2-h1 library (H1). The library contains sgRNAs targeting 2,318 druggable genes encoding targets with approved or clinical-phase drug candidates or with known bioactive drug-like small molecule binding partners, including kinases and phosphatases. There are 5 sgRNAs per gene, along with 250 non-targeting control sgRNAs (13,025 total elements). iPSCs were infected with lentivirus sgRNA library at a 0.4 multiplicity of infection (MOI) to ensure single sgRNA expression per cell, followed by puromycin selection, then differentiated into neurons. GR50 or GFP control was expressed in the sgRNA-expression neurons on day 5 of differentiation and harvested the remaining live neurons on day 14. To ensure the representative coverage of the library, neurons were maintained at >1000-fold of the library complexity after drug selection and ~500-fold at the end of the neuronal stage. The genomic DNA was extracted and libraries were constructed for high-throughput sequencing (FIG. 1C). The enrichment of individual sgRNA in surviving neurons expressing either GFP control or GR50 was compared, and the gene-level effects were calculated from five sgRNAs targeting each gene by Cas9 high-throughput maximum-likelihood estimator (casTLE) algorithm. The genes with sgRNAs enriched in surviving GR50-expressing neurons compared to the GFP control were flagged as candidates with GR-specific beneficial effects for neuron survival.

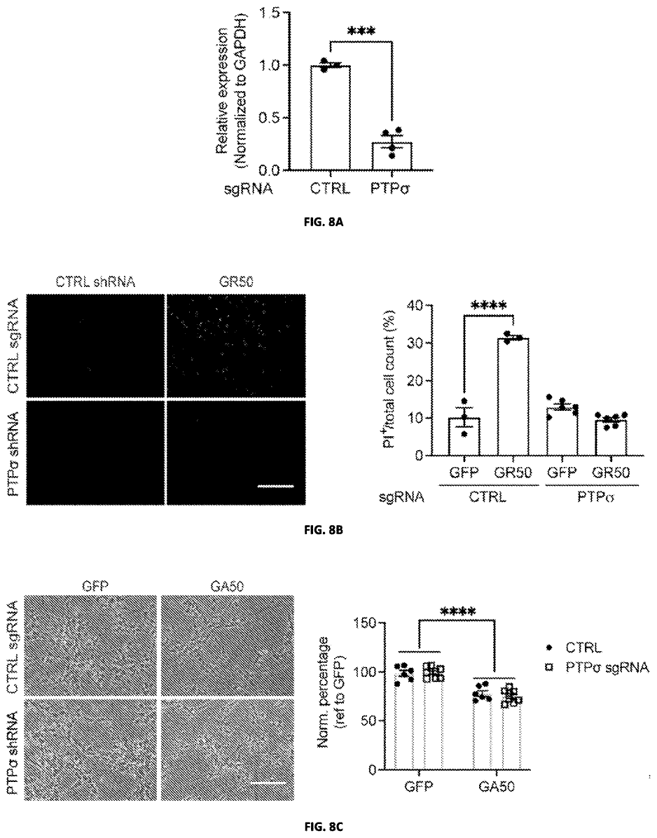

Down-regulation of the expression level of PTPσ improved the survival of GR50-expressing neurons (FIG. 1D). To confirm this finding, independent sgRNA were designed to target PTPσ for transcription repression. GR50 expression was transduced in day-5 i³Neuron expressing PTPσ or non-targeting control sgRNA and neuronal survival was examined on day 14 (FIG. 8A). iPSC lines were then generated expressing either PTPσ sgRNA or a non-targeting control sgRNA, followed by neuronal differentiation and a 7-day GR50 transduction. GR50-induced neurotoxicity was significantly ameliorated in the PTPσ knockdown i³Neurons as evidenced by increased neuron survival rate and reduced PI staining (FIGS. 1E and 8B), confirming the protective effect of PTPσ reduction. However, PTPσ downregulation did not reduce the neurotoxicity induced by GA50 expression or TDP-43 knockdown in i³Neurons (FIGS. 8C-8D). Collectively, these observations demonstrate PTPσ is a strong and specific modifier of poly-GR mediated toxicity, and its downregulation can increase the resistance of neurons to GR-induced cell death.

### Example 2

### PI3P Level is Decreased in Poly-GR Expressing Neurons

To decipher the mechanism of PTPσ mediated neuronal protection in poly-GR expressing neurons, poly-GR expression was transduced in PTPσ knockdown and control i³Neurons for 9 days. Immunofluorescence staining showed that poly-GR expression was not decreased after knocking down PTPσ (FIG. 8E), suggesting the neurons become resistant to poly-GR toxicity by altering PTPσ-associated biological processes rather than decreasing poly-GR level.

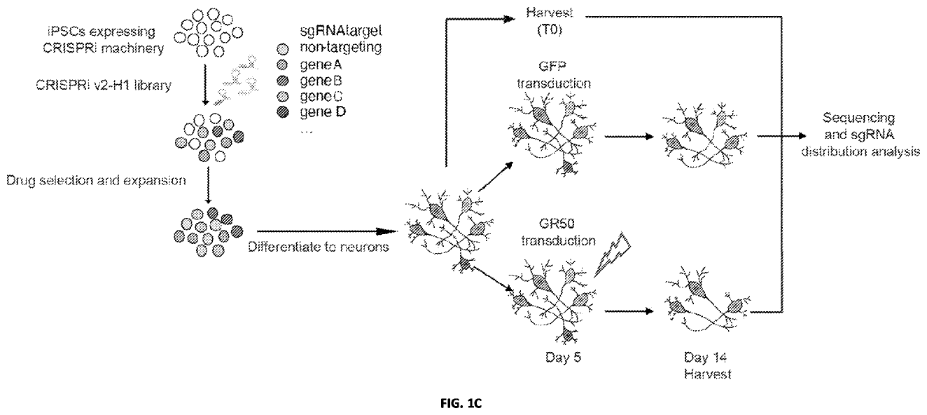

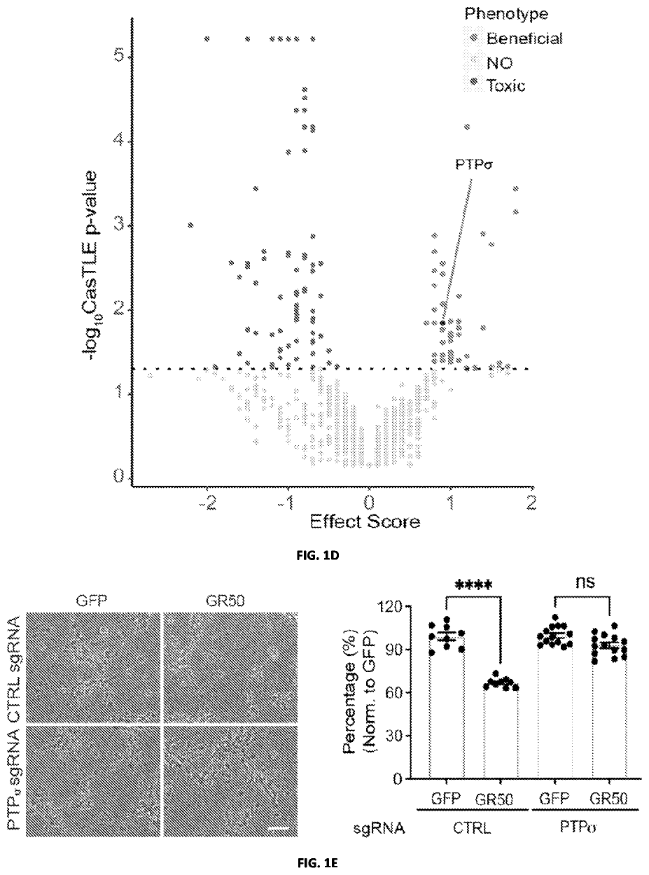

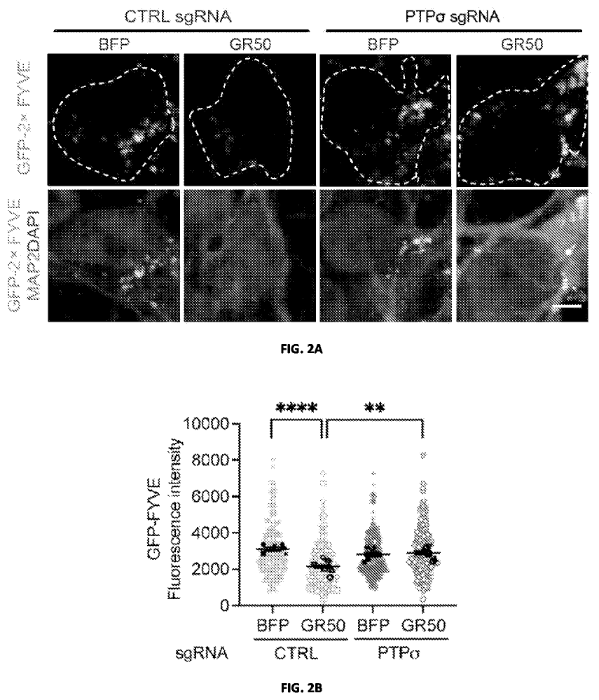

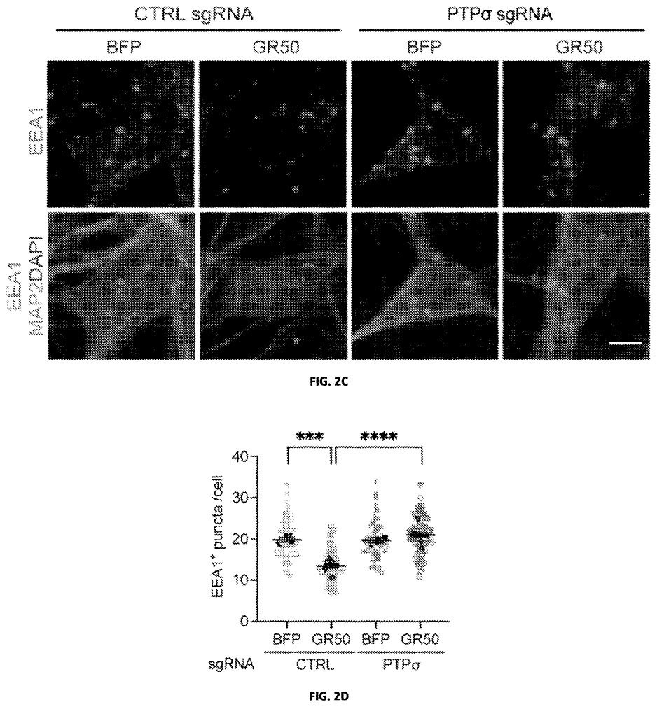

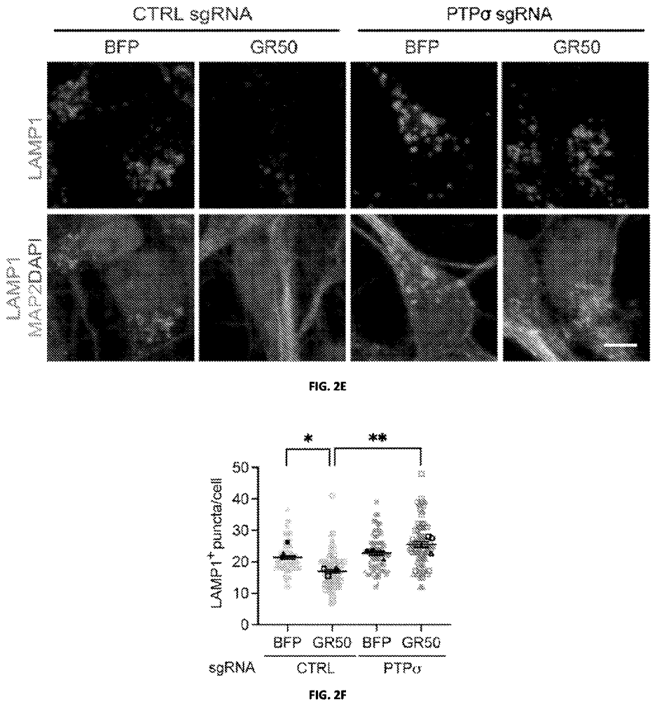

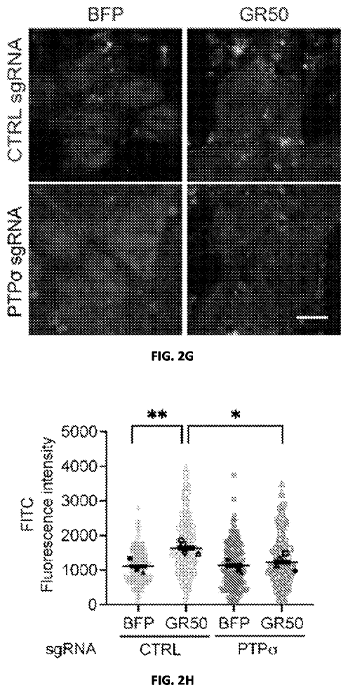

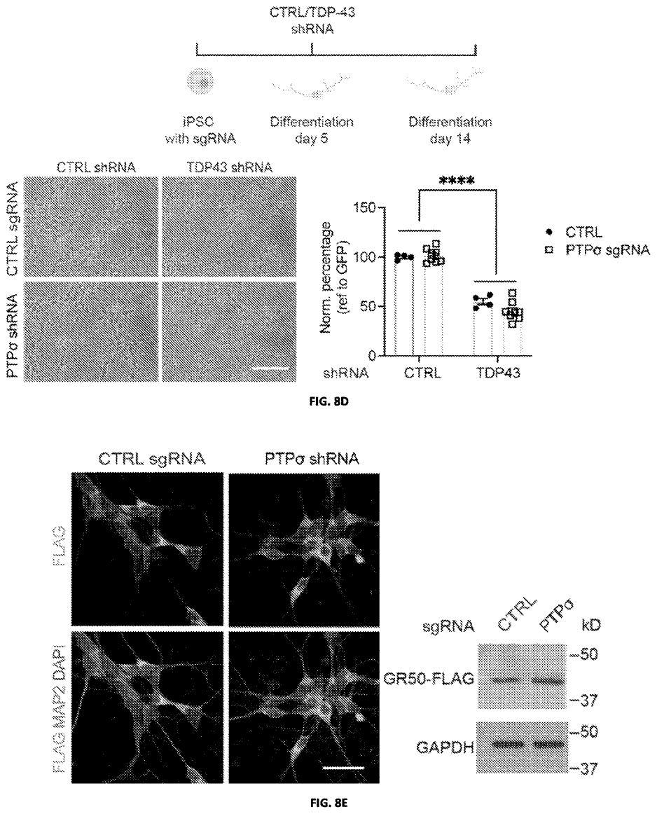

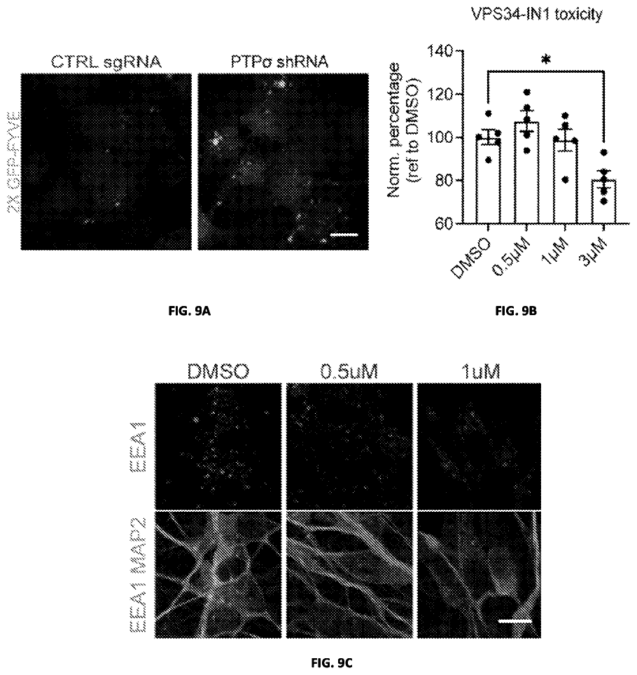

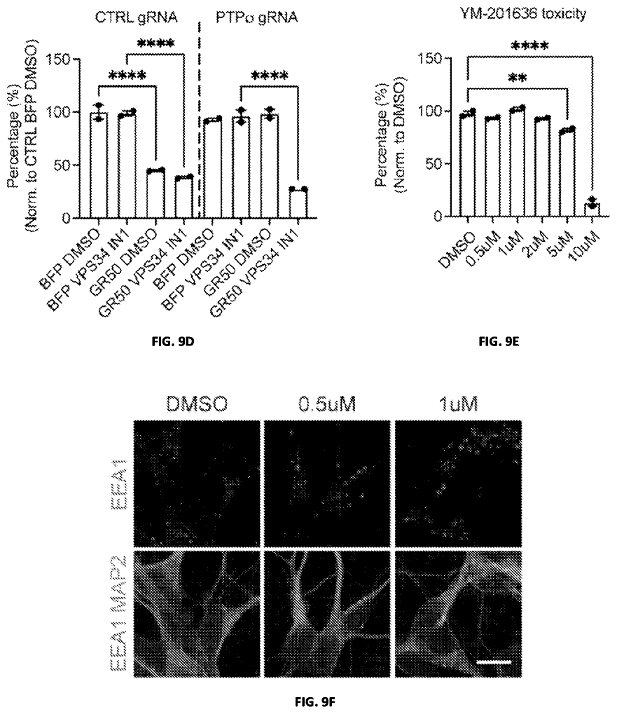

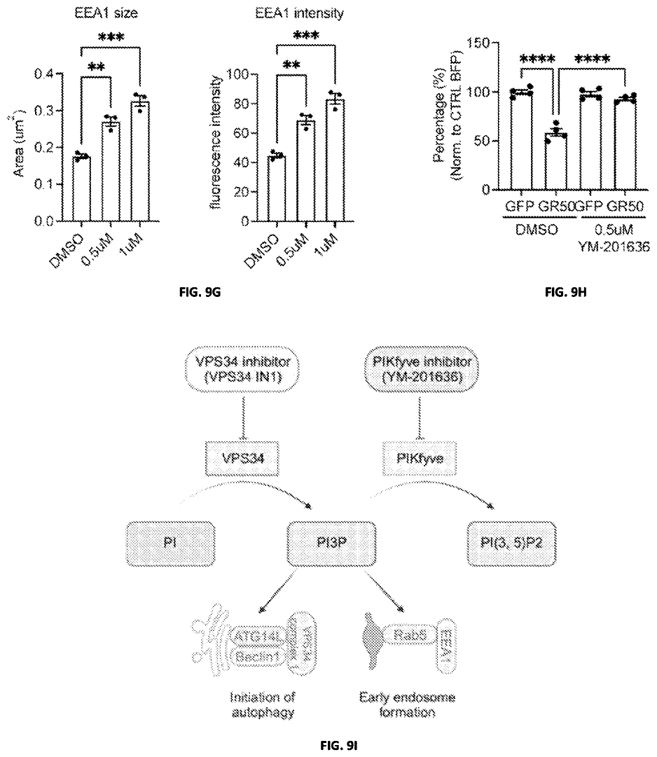

One of the major functions of PTPσ is to regulate PI3P levels. A siRNA-based screening identified PTPσ as the strongest regulator for PI3P production. To determine whether PTPσ modulates neuron survival by regulating the PI3P associated pathways, examination of whether the PI3P level is perturbed in poly-GR50 expressing neurons was studied by applying the GFP-2×FYVE reporter. The FYVE domain directly binds to PI3P, therefore the tagged GFP can indicate the PI3P level in cells. A significant increase of GFP signal was found upon knocking down PTPσ in the U2OS cells (FIG. 9A). In neurons, to rule out the possibility that massive cell death might result in alteration in PI3P level, the neurons were examined 3-days after GR50 transduction when no obvious cell death was observed (FIGS. 7G and 10A). A significant reduction of GFP fluorescent intensity was detected in poly-GR expressing neurons, suggesting the reduced PI3P level in the cells. When knocking down PTPσ, the GFP fluorescent intensity increased to the comparable level of BFP expressing neurons, indicating the elevation of PI3P level (FIG. 2A).

### Example 3

### PI3P Elevation Contributes to PTPσ Knockdown-Mediated Neuroprotection

To inhibit the synthesis of PI3P such that the rescue effect would be abolished, PTPσ knockdown GR50 expressing i³Neuons were treated with 0.5 μM VPS34 inhibitor (VPS34-IN1) (FIGS. 9B-9C). VPS34 specifically catalyzes PI3P synthesis from PI to PI3P. Two days after the treatment, while a robust rescue effect of PTPσ knockdown in poly-GR expressing neurons was still observed, the effect was completely diminished after VPS34-IN1 treatment (FIG. 9D), suggesting PI3P elevation contributes to the neuroprotection. Additionally, the cells were treated with a PIKFYVE inhibitor (YM-201636) which can boost PI3P levels by inhibiting the conversion of PI3P to PI (3,5) P2. The data showed a robust rescue effect of YM-201636 in poly-GR expressing neurons (FIG. 9H), overall suggesting the elevation of PI3P level is beneficial for survival of poly-GR expressing neurons.

### Example 4

### PI3P Elevation Rescues Deficits of Endo-Lysosomal System in Poly-GR Expressing Neurons

During early endosome formation, Rab5 recruits EEA1 to the early endosomal membrane via local PI3P, thus EEA1 expression is considered a secondary indicator of PI3P level. To demonstrate the alternation of EEA1 expression is an early event before cell death, poly-GR expression was transduced for 3 days. Immunofluorescence staining showed a significant reduction of EEA1 puncta in poly-GR expressing neurons, suggesting the reduction of EEA1⁺ early endosome formation and the reduced PI3P level in cells (FIG. 2B). However, an alteration of Rab5 level was not detected between GFP and poly-GR expressing neurons (FIG. 10B), suggesting it is likely that Rab5 fails to recruit EEA1 to early endosomal membrane due to the reduction of PI3P level in poly-GR expressing neurons. EEA1⁺ endosome formation was increased upon knocking down PTPσ in poly-GR expressing neurons (FIGS. 2C-2D).

To determine if the reduction of EEA1 is specific to poly-GR, poly-GA expression was transduced for 3 days or TDP-43 was knocked-down for 5 days before significant cell death was observed. No significant reduction of EEA1 fluorescence intensity, EEA1 puncta number and size were observed in poly-GA expressing (FIG. 10C) and TDP-43 knockdown neurons (FIG. 10D) comparing to the BFP expressing control or control shRNA transduced neurons. The evidence suggested the reduction of EEA1⁺ early endosomal formation is specific to poly-GR expression under the current condition.

Early endosome goes through transportation and partially becomes late endosome/lysosome, which facilitates protein degradation and microenvironment homeostasis. The expression of the lysosomal marker LAMP was examined in neurons. There was a significant decrease of LAMP1 puncta in poly-GR expressing neurons which can be rescued by knocking down PTPσ (FIG. 2C). To detect lysosomal deficits, FITC-Dextran was incubated with i³Neurons for 16 hours. FITC signal was captured after 2 hours chase process. The FITC fluorescence will become brighter if the environment within lysosome becomes less acidic indicating impaired lysosomal function. Meanwhile, if the lysosomal membrane is not intact, the signal is diffused to the cytosol. Lysosomal function was examined in poly-GR expressing neurons with 3 days-transduction. At this time point, EEA1 reduction was presented without apparent cell death (FIG. 10F). Indeed, increased fluorescent intensity in lysosomes along with cytosolic green fluorescence was observed in poly-GR expressing neurons compared to the BFP-expressing neurons (FIG. 2D). Moreover, the fluorescence intensity was significantly decreased in the PTPσ knockdown poly-GR expressing neurons. These findings show that poly-GR impairs endo-lysosomal pathway in neurons which can be rescued by knocking down PTPσ.

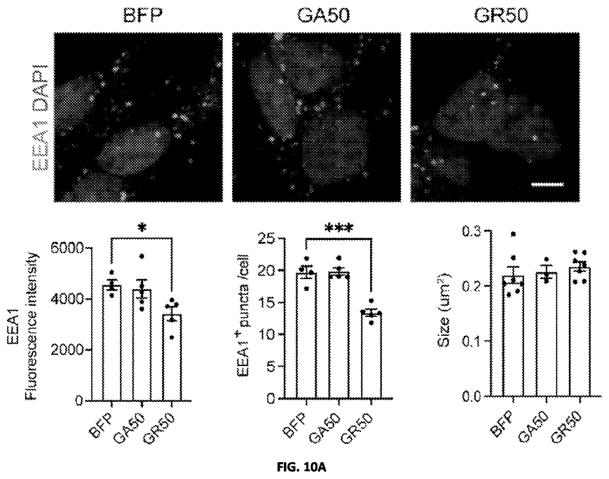

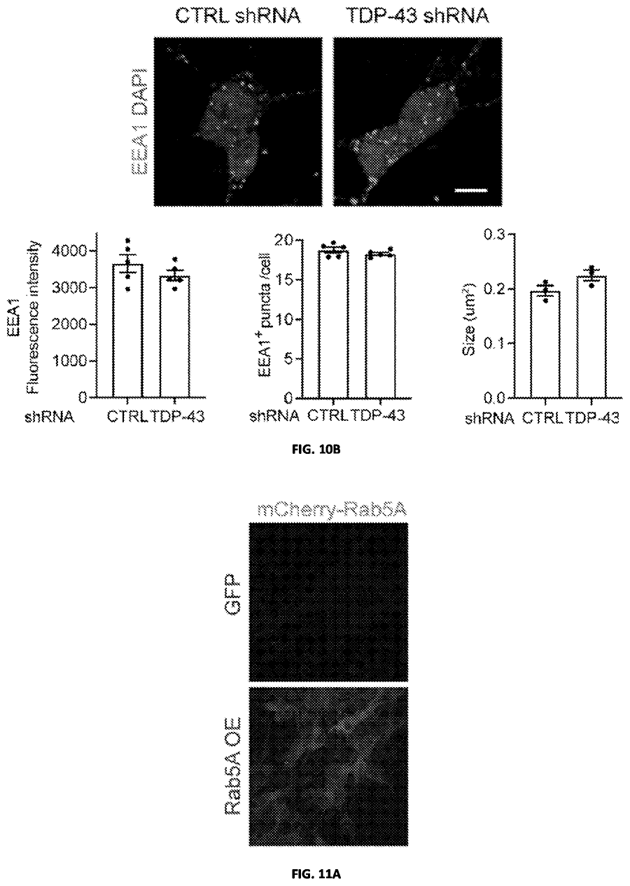

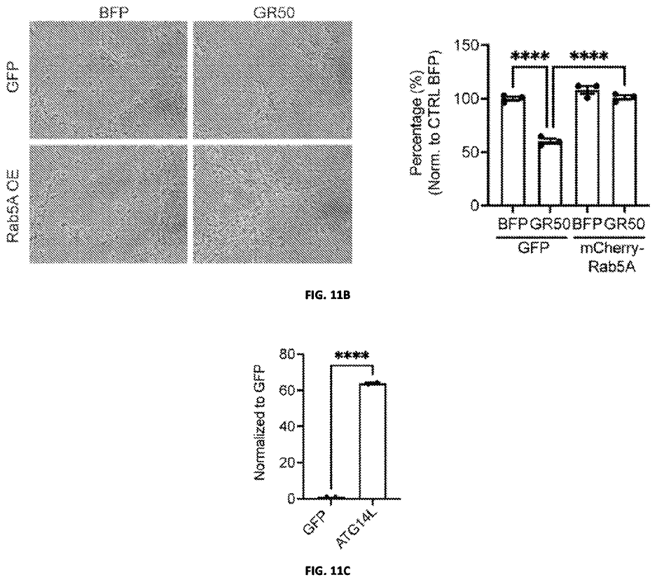

To distinguish the dominate source of the rescue effect in the poly-GR expressing neurons, an active mutation of Rab5 (Q79L) which results in the recruitment of PI3P to membrane and enlarged early endosome was overexpressed (FIG. 11A). Improved neuron survival was observed after Rab5 (Q79L) overexpression (FIG. 11B). Interestingly, overexpression of ATG14L, a key component of the autophagy initiation complex, did not improve the survival of poly-GR expressing neurons, indicating the rescue effect of elevating PI3P in the poly-GR expressing neurons might be more associated with endo-lysosomal pathway than autophagy, even though PI3P level is important for both pathways (FIGS. 11C-11D).

### Example 5

### PTPσ Knockdown is Sufficient to Ameliorate the Deficits of Endo-Lysosomal System and Glutamate-Induced Neurotoxicity in C9ORF72-ALS Patient iPSNs

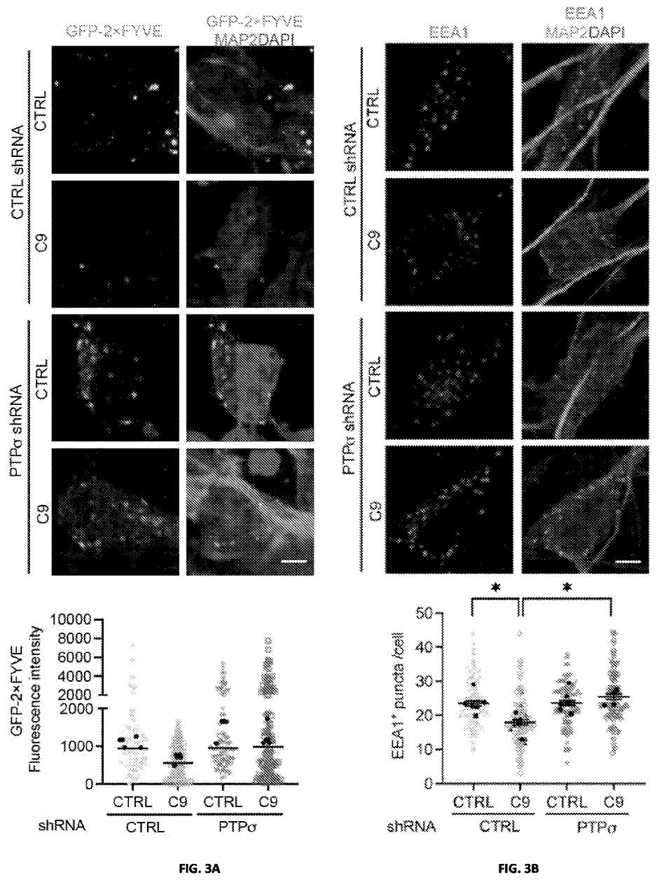

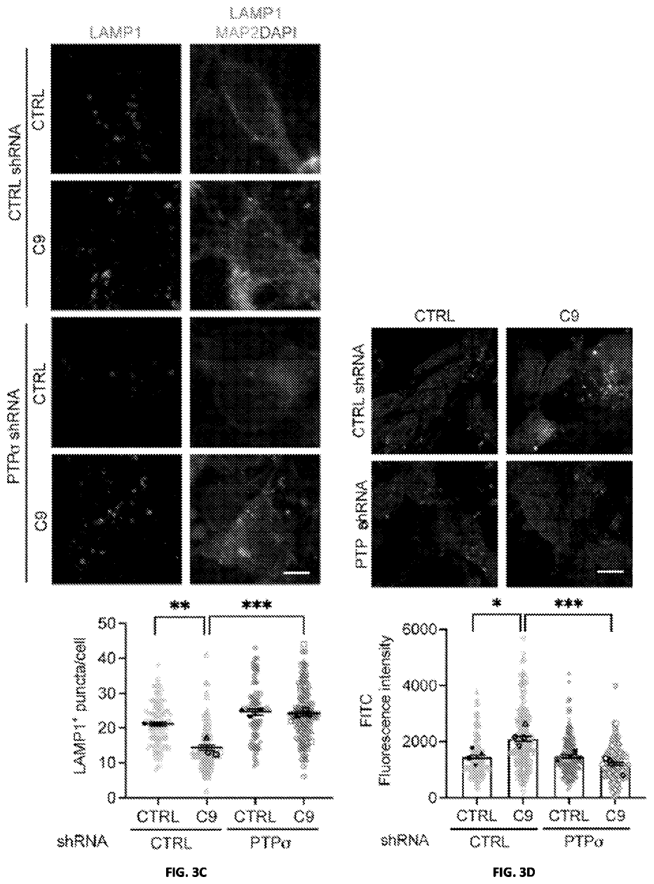

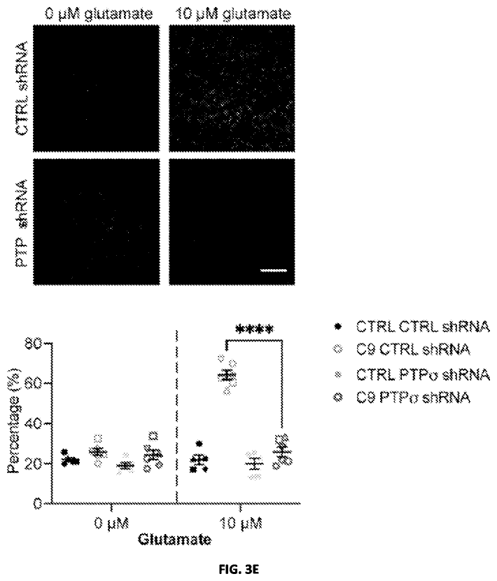

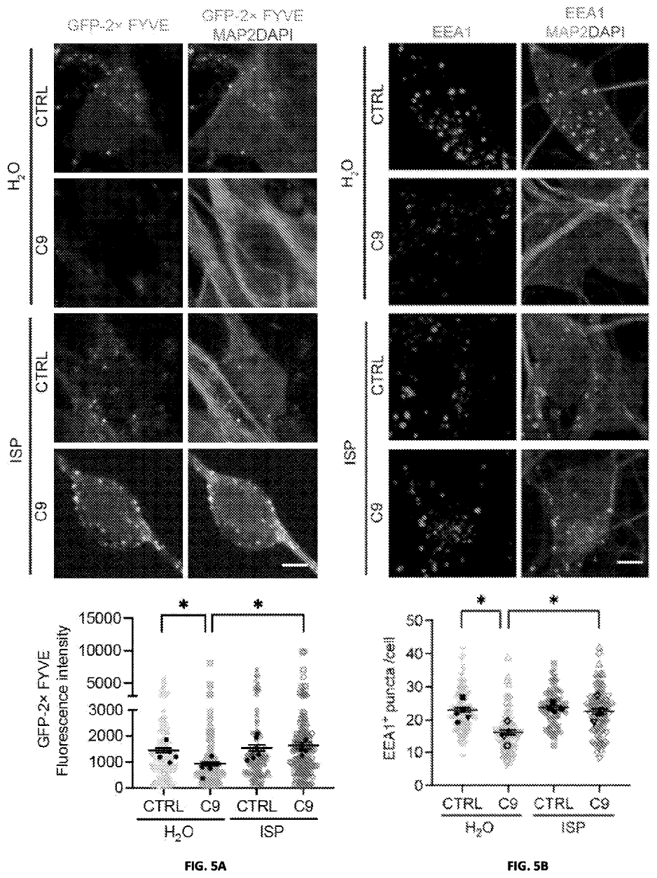

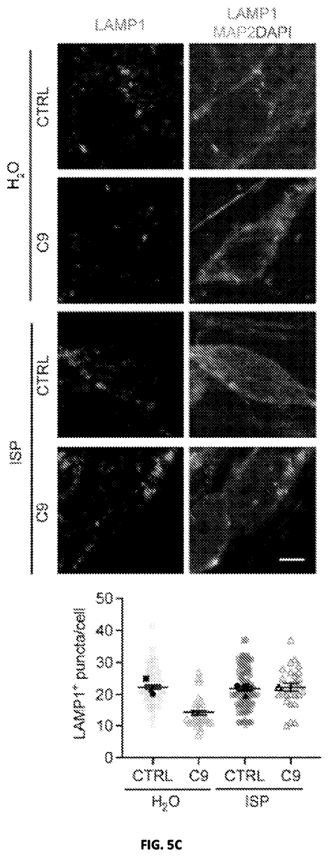

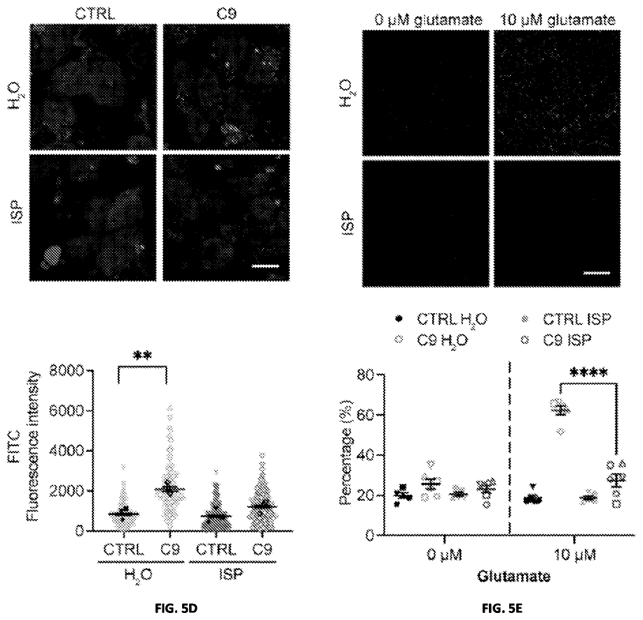

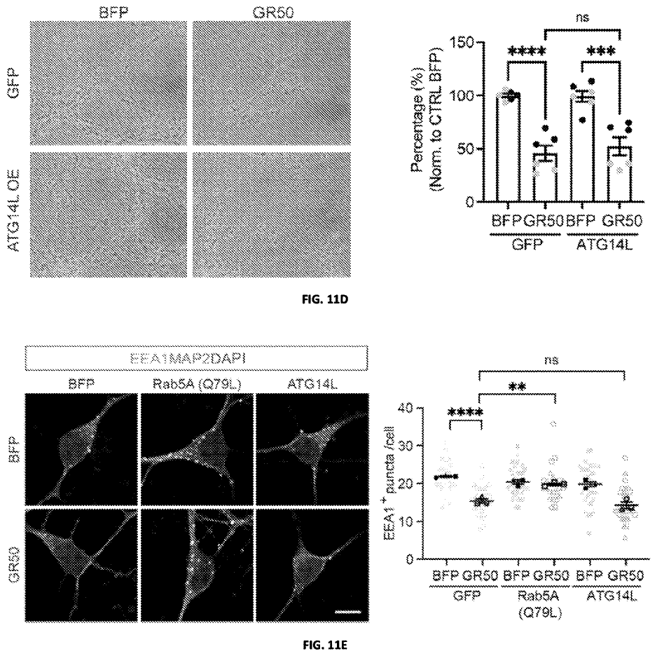

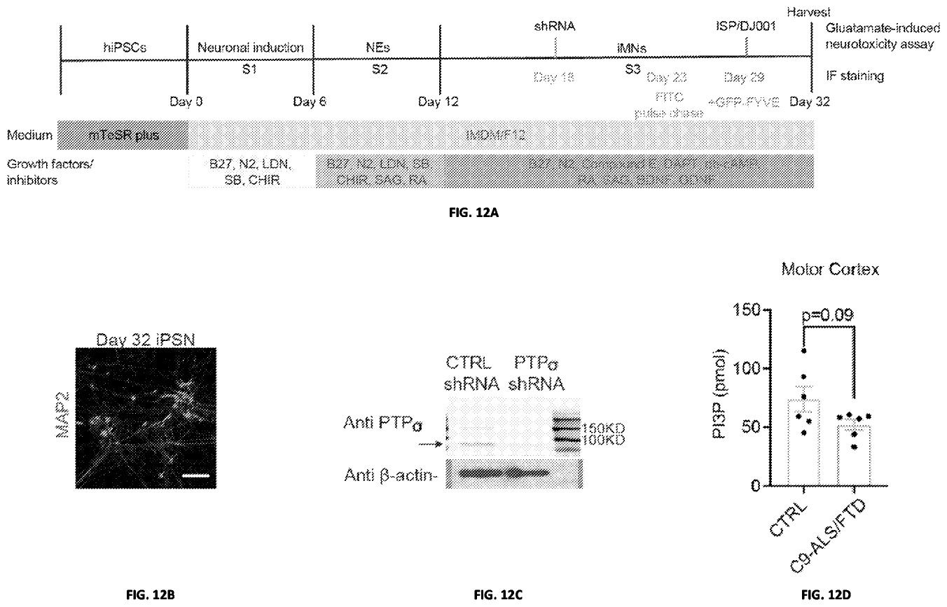

DPRs mediated toxicity is one aspect of the C9ORF72-ALS/FTD pathogenesis. In order to further investigate the role of PI3P level in the disease onset and progress, especially in the endo-lysosomal system, the abnormalities in the C9 patient iPSNs were examined. A direct induced motor neuron protocol, which yields a reproducible population of spinal neurons containing about 30% Islet-1 positive lower motor neurons, was used (FIGS. 12A-12B). An shRNA targeting PTPσ was designed and expressed in day-18 neurons via lentiviral transduction. GFP-2×FYVE reporter was transduced by lentivirus on day 30. When harvesting at day 32, the endogenous PTPσ level was significantly downregulated compared to the non-targeting control (FIG. 12C). No significant toxicity of control and PTPσ shRNA was observed. GFP signal and EEA1 were examined by immunofluorescence (IF) staining. The results showed a significant GFP-2×FYVE and EEA1 signal reduction in the C9-iPSNs, which are ameliorated after PTPσ knockdown (FIGS. 3A-3B). The reduction of lysosome was also detected in the C9-iPSNs (FIG. 5C). Lysosomal function was further determined by the FITC pulse-chase assay. As C9-iPSNs intend to form clusters and experience higher stress level in the 32 days culture, it is difficult to distinguish if the lysosomal function impairment is an early event. Therefore, shRNA was transduced on day 18 and the FITC fluorescent intensity was measured in day 23 neurons. Significant increases of averaged fluorescent intensity were seen in the C9-iPSNs, suggesting increased pH in the lysosome. However, the FITC intensity decreased upon PTPσ knockdown, indicating PTPσ down-regulation is beneficial for the correction of lysosomal pH (FIG. 3D).

Glutamate toxicity contributes to the disease onset/progression in ALS due to the loss of glutamate transporter 1 (EAAT2). C9-iPSNs have been found to be more vulnerable to glutamate treatment than controls. Targeting G4C2 repeats, DPRs can significantly reduce the glutamate-induced neurotoxicity, implicating the improvement of neuronal function. To evaluate the effect of PTPσ knockdown to the physiology of C9-iPSNs, glutamate-induced excitotoxicity assays were performed and the percentage of cell death (PI⁺) was quantified after the treatment with or without PTPσ knockdown. Treatment with 10 μM glutamate led to the increased PI⁺ dead cells in the control shRNA transduced C9-iPSNs, whereas cell death was significantly reduced in PTPσ knockdown C9-iPSNs (FIG. 3E). Taken together, the evidence supports PTPσ knockdown as beneficial for homeostasis of endo-lysosomal system, neuronal function, and survival in the C9-iPSNs.

### Example 6

### Endo-Lysosomal Impairment in C9 Patient Postmortem Tissue

The data above demonstrates that PI3P level and EEA1 puncta were decreased in poly-GR expressing neurons and C9-iPSNs. To determine if the deficit is presented in C9 patients the aging non-FTD control, non-C9 FTD and C9 ALS/FTD patient postmortem brains were obtained and lipids were extracted and PI3P levels were measured by a non-radioactive ELISA assay. In the temporal cortex, there was a close to statistically different (two tailed Student's t test, p=0.054) reduction of PI3P levels of C9 patients compared with non FTD control samples. Meanwhile, no difference was found between the non-FTD and non-C9 FTD controls, suggesting the alternation might be specific to C9-ALS FTD patient (FIG. 3F). A similar trend was also found in the motor cortex of C9-ALS/FTD patient brains (FIG. 12D). EEA1 was stained by IF in the temporal cortex of C9 patients. There were fewer EEA1 puncta in the neurons of C9 patient tissues with significantly lower fluorescent intensity within neurons (FIG. 3G).

### Example 7

### PTPσ Inhibitor Rescues Endo-Lysosomal Defects and Improves Survival of Poly-GR/PR Expressing Neurons and C9-iPSNs

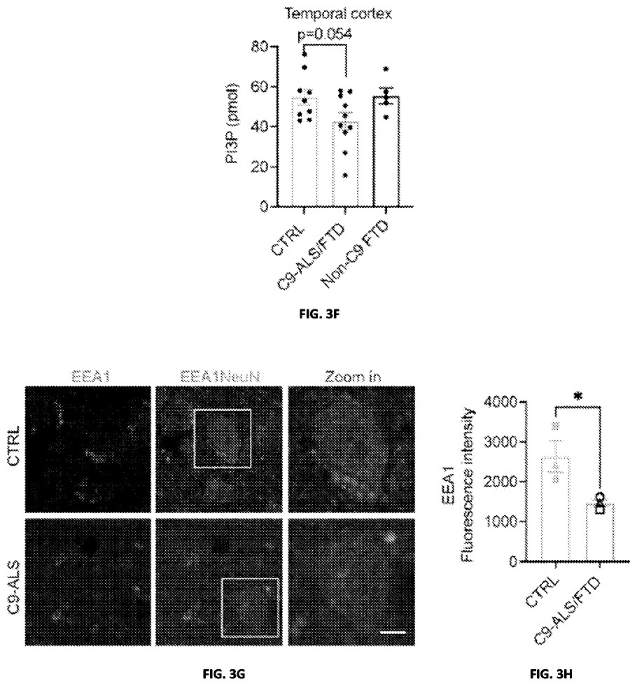

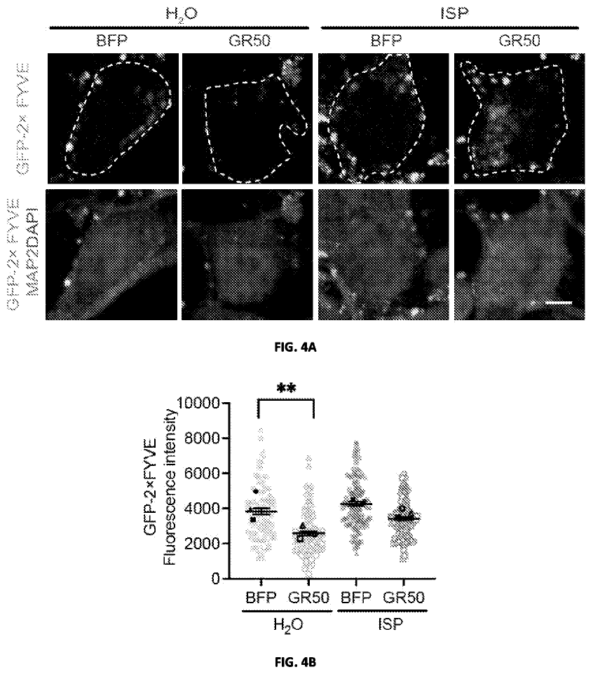

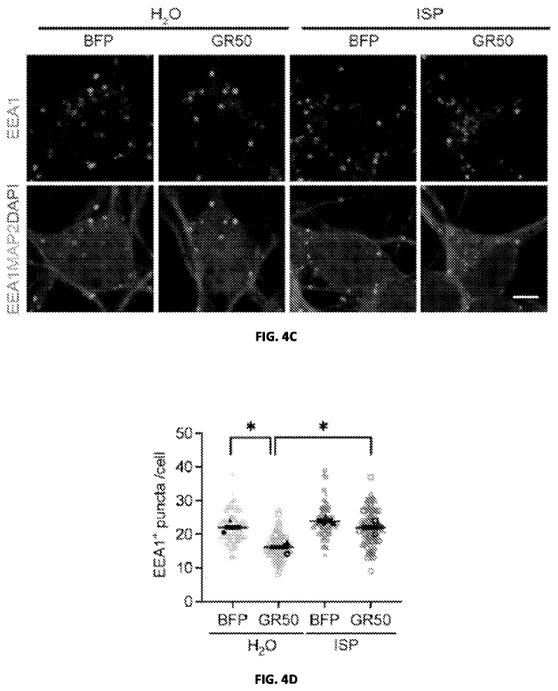

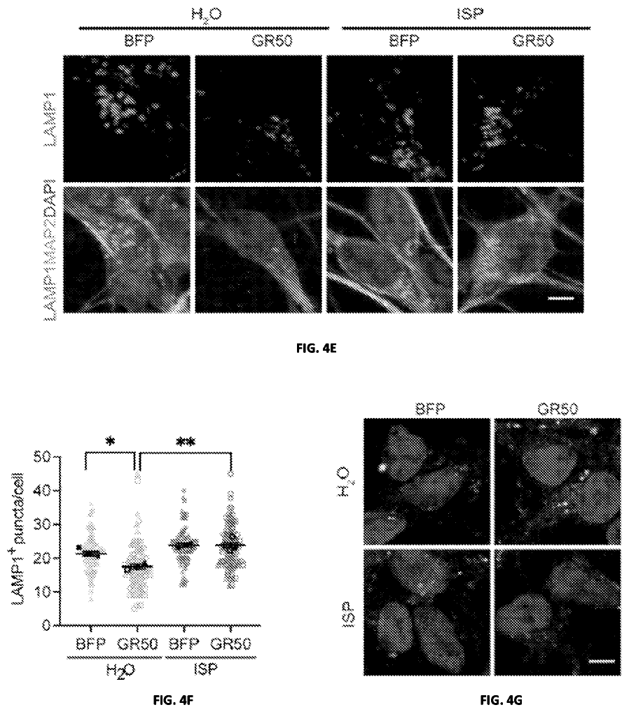

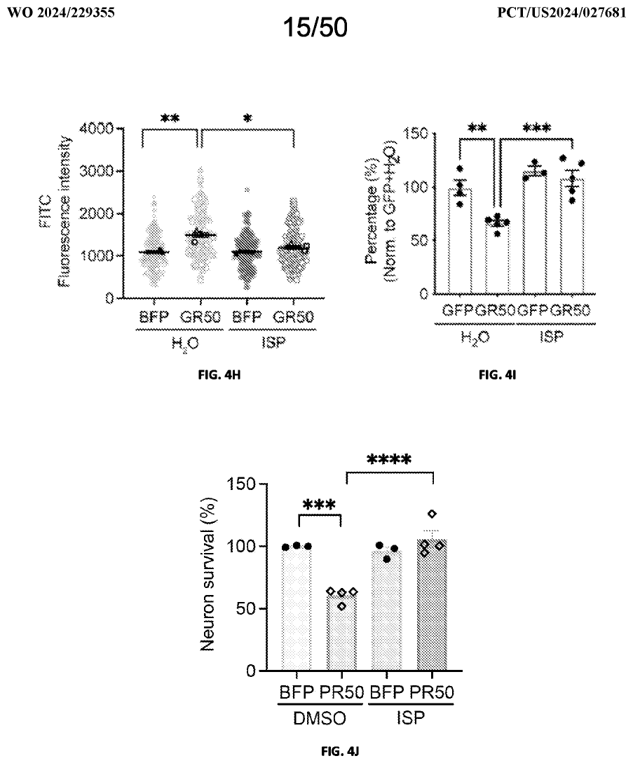

To determine if PTPσ inhibitors can recapitulate the rescue effect of PTPσ knockdown, poly-GR expressing neurons were treated with ISP, a cell-permeable, TAT-conjugated peptide derived from the PTPσ wedge domain that binds to PTPσ. The TAT domain enables ISP to cross the blood-brain barrier (BBB). ISP treatment, 3-days 0.5 μM, significantly increased PI3P level detected by GFP-2×FYVE reporter (FIG. 4A), EEA1⁺ early endosome (FIG. 4B), LAMP1⁺ lysosome number (FIG. 4C) and improved lysosomal function (FIG. 4D), therefore promoting the survival of poly-GR and poly-PR expressing neurons (FIG. 4E, 4J).

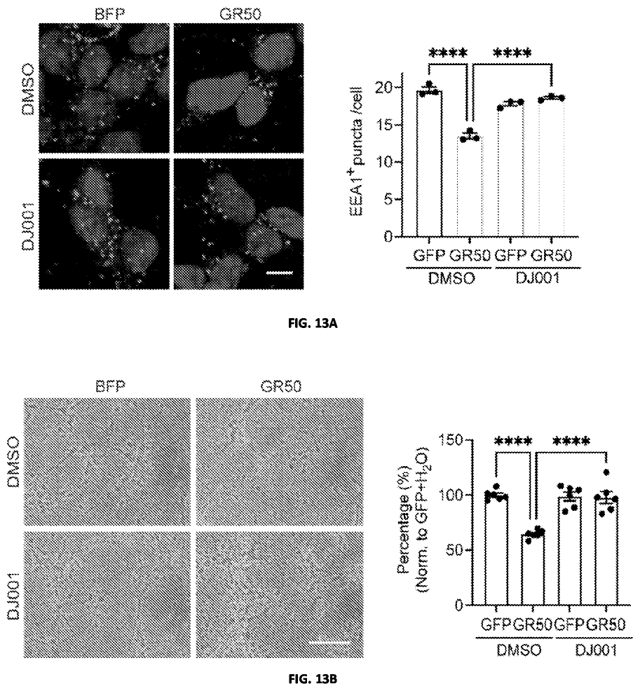

DJ001 is a highly specific inhibitor of PTPσ which can promote hematopoietic stem cell regeneration in vitro and in the mouse model, although it is unknown if it can penetrate BBB. Similar to the effect of ISP, DJ001 treatment also significantly increased the formation of EEA1⁺ early endosome and improved poly-GR/PR expressing neuron survival (FIGS. 13A-13D).

Control and C9-iPSNs were treated with ISP on day 29 and cells were harvested on day 32 to evaluate the impact of ISP in C9-iPSNs. Similar to the effect of PTPσ knockdown, ISP treatment increased PI3P level (FIG. 5A), EEA1⁺ puncta (FIG. 5B), LAMP1⁺ signal (FIG. 5C), restored lysosomal function (FIG. 5D), and rescued glutamate-induced neuroexcitotoxicity in the C9 iPSNs (FIG. 5E). Similar rescue effects were observed after DJ001 treatment (FIG. 5F), supporting PTPσ inhibition as a potential strategy to rescue endo-lysosomal defects and improve survival of poly-GR expressing neurons and C9-iPSNs.

### Example 8

### ISP Administration Rescues Cellular and Behavioral Phenotypes in GR100 Mice

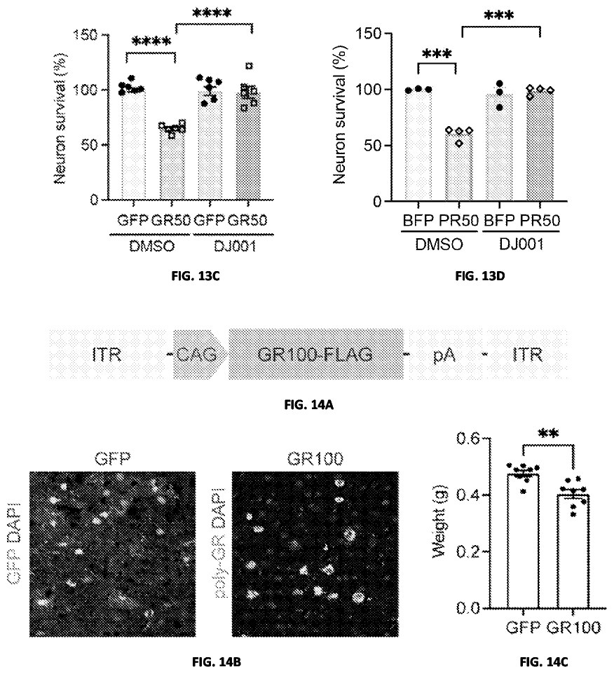

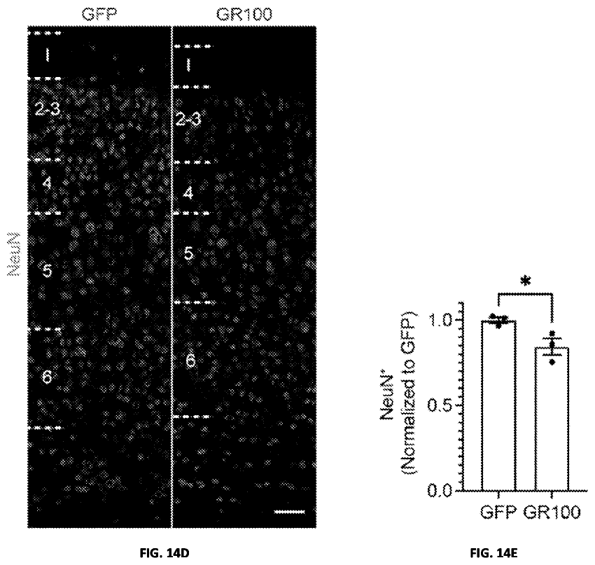

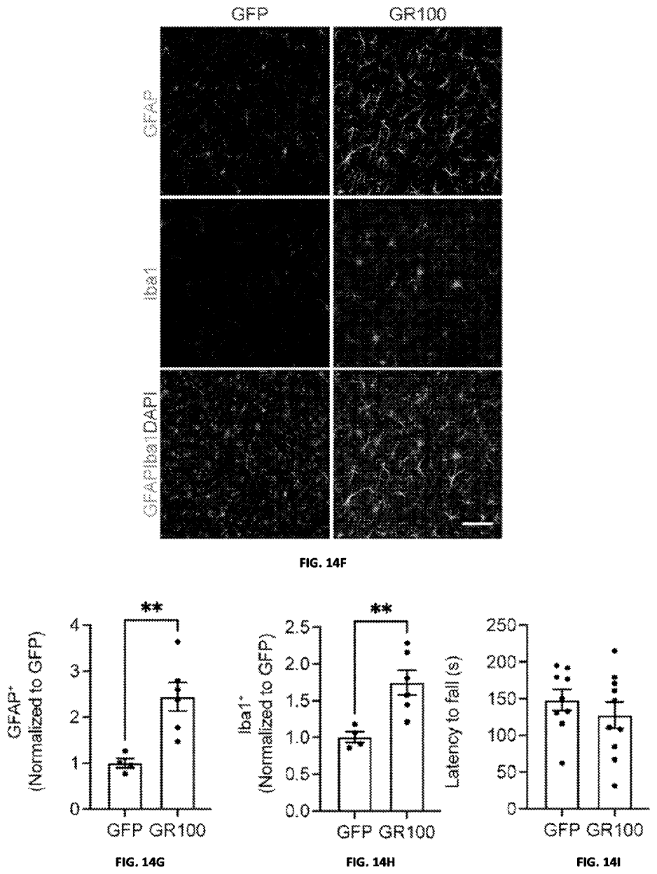

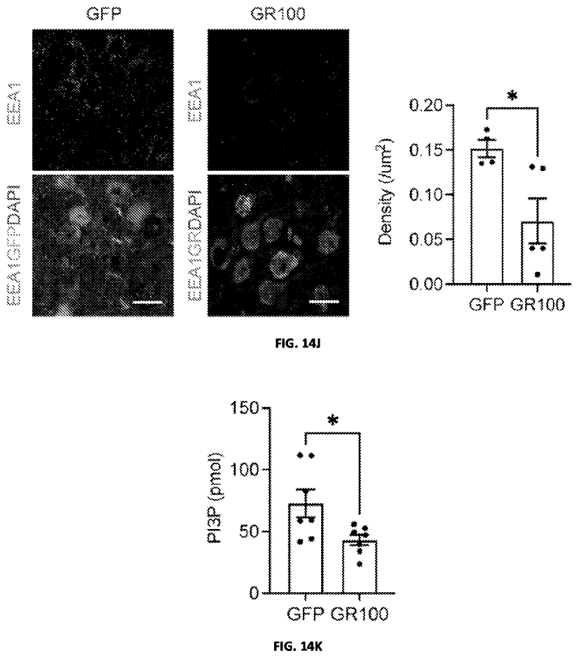

PTPσ inhibition can significantly improve poly-GR expressing neuron survival in vitro, suggesting its potential as a therapeutic target. The successful rescue of the cell survival, endo-lysosomal defects by ISP treatment and the validated BBB penetrate capability makes it a good candidate for further validation. To validate the rescue efficacy of ISP in vivo, a GR100 mouse model was established by AAV delivery. AAV-PHP.eB expressing 100 repeats of GR dipeptides (GR100) under CAG promoter (FIG. 14A) were delivered into the brains of P0 neonatal mice (C57BL/6) via intracerebroventricular (i.e.v) injection. AAV-GFP was used as the control.

One and a half months after virus injection, robust GFP or poly-GR expression was detected in mouse brains (FIG. 14B). GR100 mice showed significantly reduced brain weight (FIG. 14C), neuronal loss (FIGS. 14D-14E), astrogliosis and microgliosis (FIGS. 14F-14H). No obvious motor function defect was found in the rotarod experiment (FIG. 14I). A significant reduction of EEA1⁺ puncta was detected in GR100 mouse brains. PI3P levels in the brain were measured by ELISA assay. A significantly decreased level of PI3P was seen in the GR100 mice compared to GFP control animals (FIG. 14K).

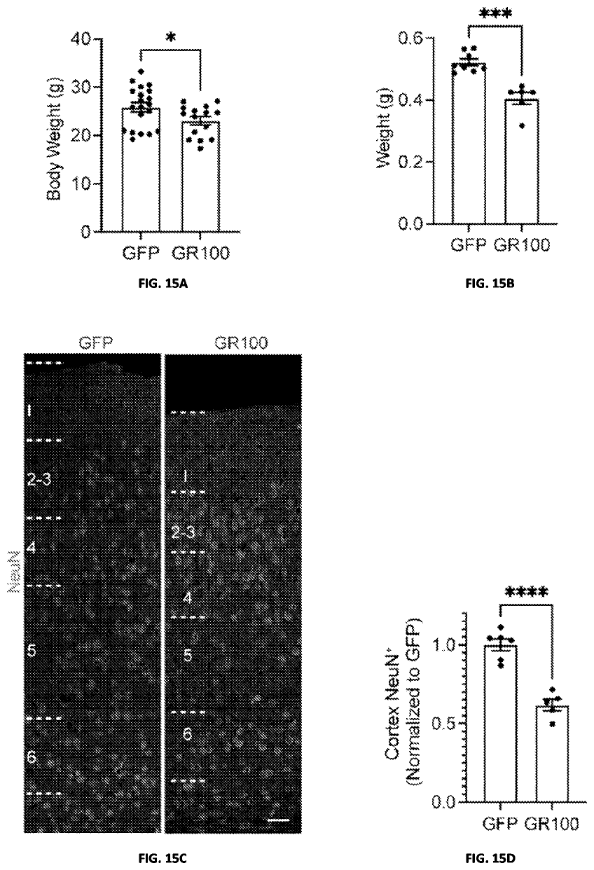

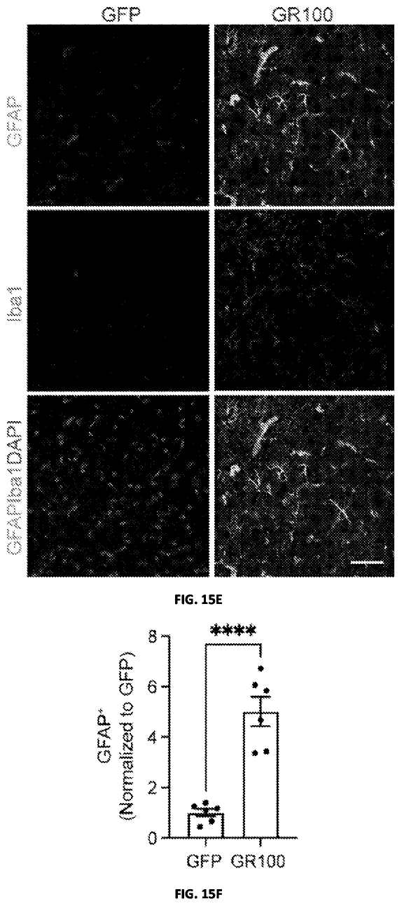

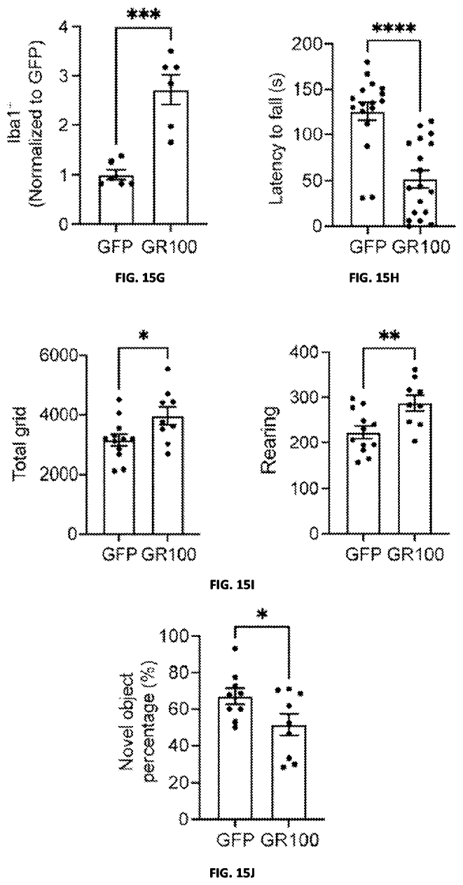

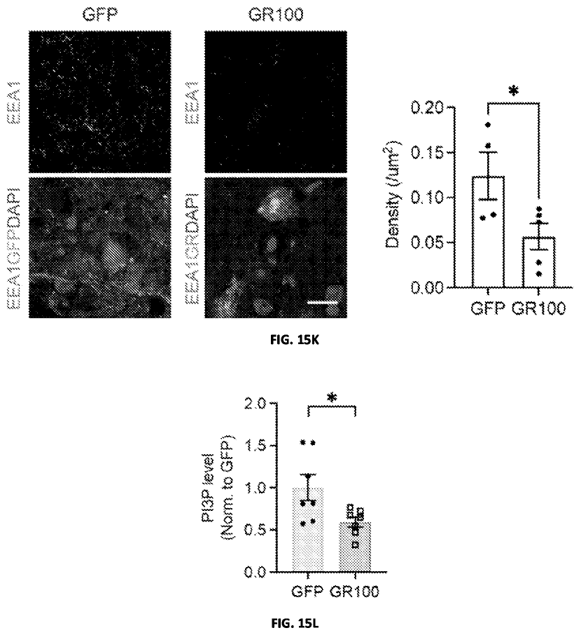

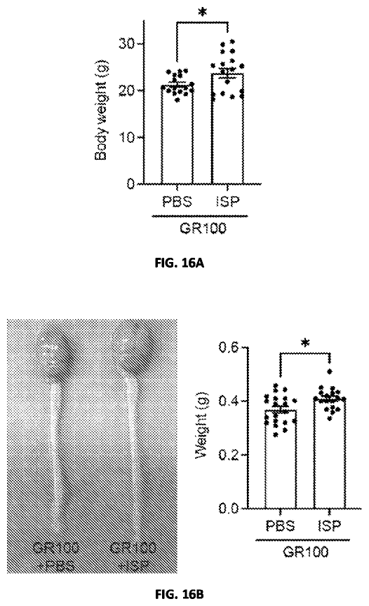

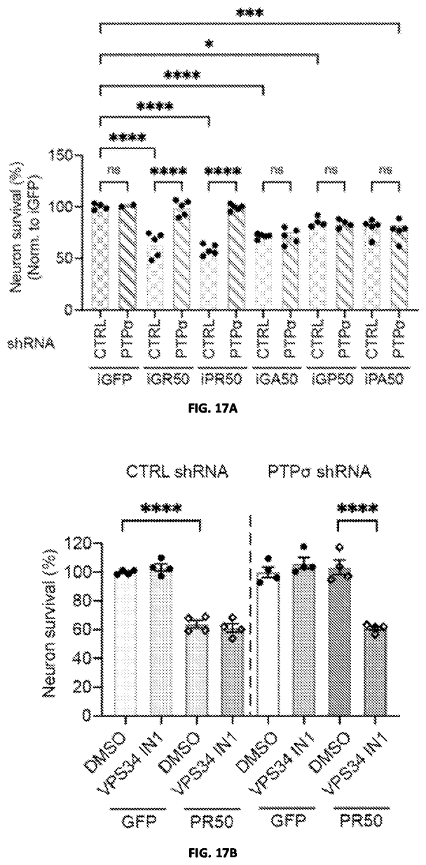

Three months after injection, the average body weight and brain weight was decreased in GR100 mice compared to GFP control (FIGS. 15A-15B). NeuN⁺ cells were further decreased compared to 1.5 months old mice (FIGS. 15C-15D), whereas GFAP⁺ and Iba1⁺ cells increased more in the GR100 mice brains at this time point, indicating the progression of neuronal loss and neuroinflammation (FIGS. 15F-15G). Besides the cellular phenotypes, GR100 mice also showed decreased latency to fall in the accelerating rotarod experiment (FIG. 15H), and increased horizonal and vertical movement in the open field (FIG. 15I). In the novel object recognition test, the GR100 mice spent less time exploring a novel object (FIG. 15J), indicating cognition defects. These pathological and behavioral phenotypes are consistent with the GFP-GR100 mice and inducible GR80 mice previously reported, demonstrating the GR100 mouse model was suitable for testing the GR toxicity modifiers in vivo. Immunofluorescence staining also showed a reduction of EEA1 in GR100 mice, suggesting a long-lasting impaired early endosome formation in vivo (FIG. 15K).

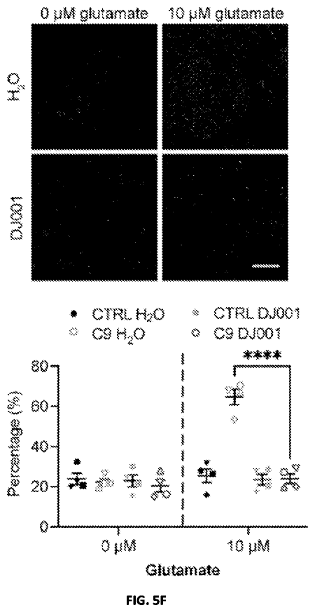

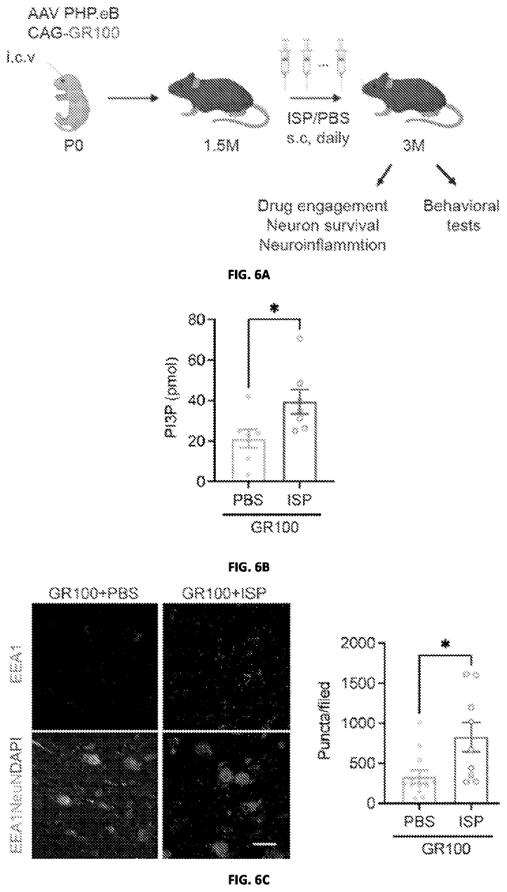

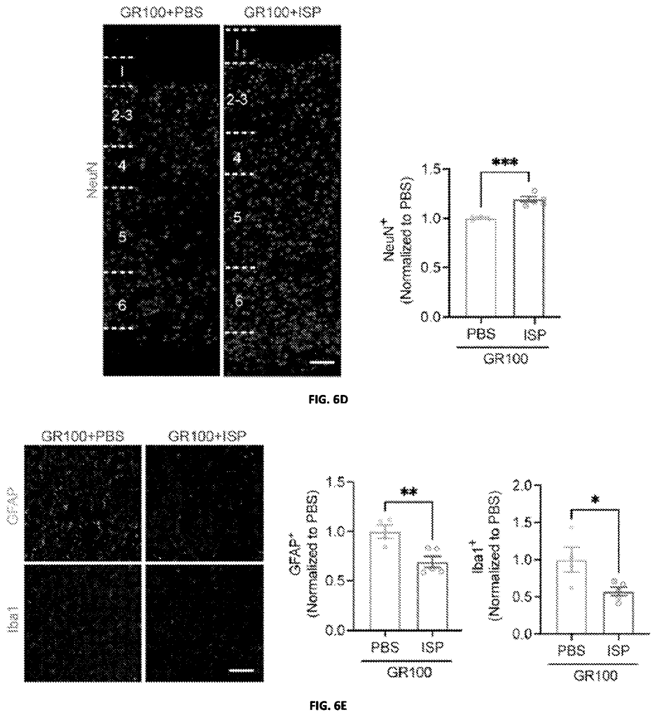

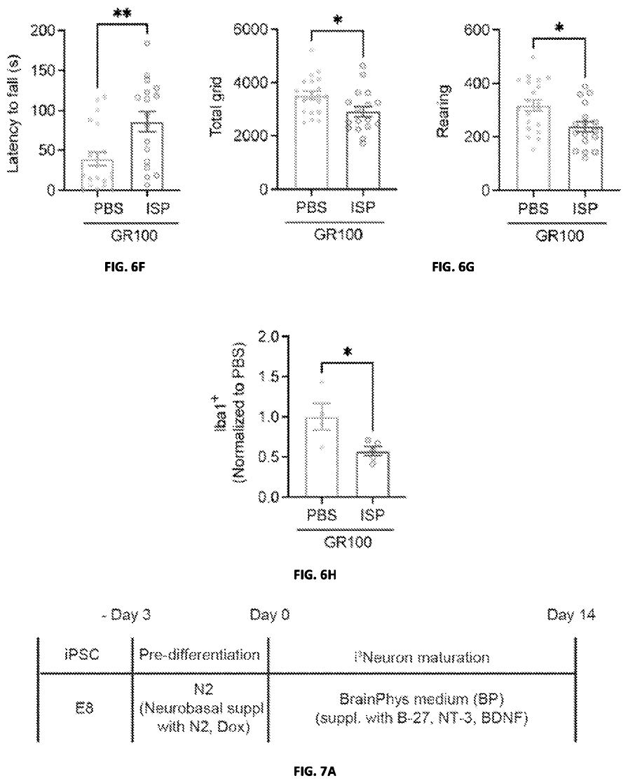

To test the efficacy of ISP administration in GR100 mice, AAV PHP.eB-GR100 virus was first delivered to P0 C57B/6 mice. ISP, 5 μg in 100 μL PBS, or PBS was injected daily starting from 1.5 months after virus delivery via subcutaneous (s,c) injection till 3 months. Behavioral tests were performed, followed by brain harvesting (FIG. 6A). The engagement of ISP in vivo was confirmed as PI3P level and EEA1 expression are increased in the ISP-treated GR100 mouse brains (FIGS. 6B and 6C).

The body weight and brain weight of the ISP-treated GR100 mice was increased compared to the PBS-injected mice (FIGS. 16A-16B). Excitingly, NeuN⁺ cells were increased with decreased GFAP⁺, and Iba1⁺ cells in the cortex of ISP-treated GR100 mice compared to PBS-treated GR100 mice, indicating less neuronal loss and lower level of neuroinflammation occurs (FIG. 6E). Moreover, the ISP-treated GR100 mice showed increased latency to fall in accelerating rotarod assay, reflecting improvement of motor function (FIG. 6F). The hyperactivity due to GR100 expression was ameliorated after ISP treatment (FIG. 6G). The mice also performed better in the novel object recognition test, indicating ISP administration was beneficial for cognition (FIG. 6H). Taken together, chronic ISP administration was well-tolerant without apparent toxicity and can rescue cellular and behavioral phenotypes in GR100 mice.

### Example 9

### PTPσ is a Strong Modifier of Poly-PR Mediated Neurotoxicity

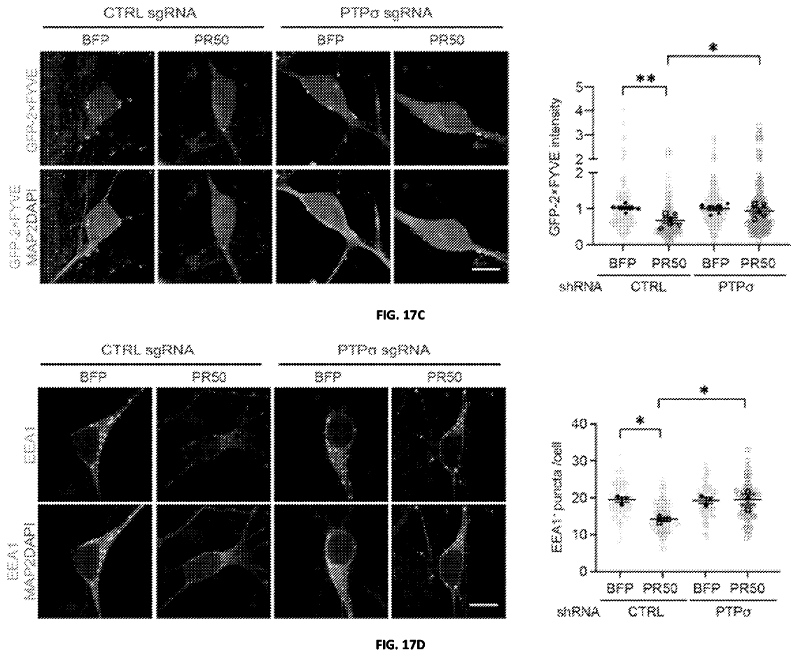

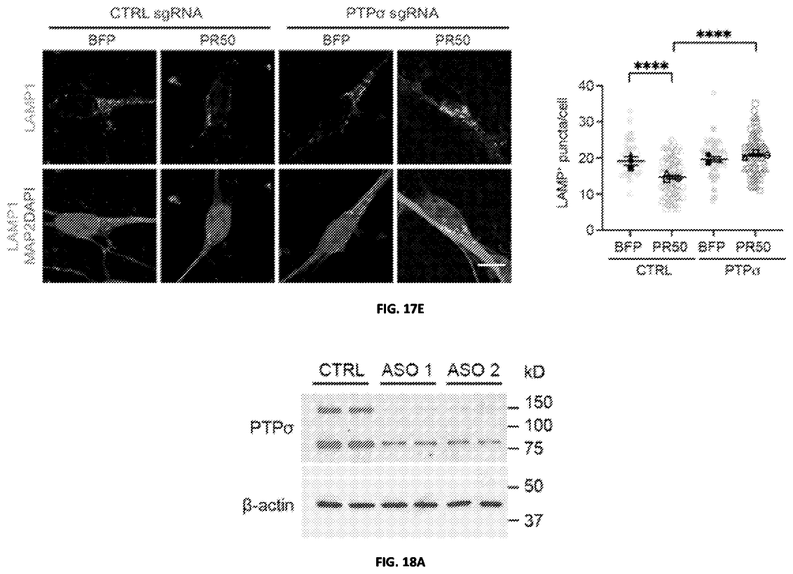

To investigate whether PTPσ could also modulate the toxicity mediated by the antisense R-DPR, poly-PR, doxycycline-inducible PR50 with C-terminus FLAG-tag was transduced, allowing DPR expression for 7 days after neuron differentiation. In line with previous findings, inducible GR50 expression led to approximately 50% reduction in neuron survival (FIG. 17A). Comparable to GR50, PR50 also resulted in significant neurotoxicity as expected, while other DPRs exhibited less toxicity (FIG. 17A). Knocking down PTPσ robustly rescued neurons from GR50 and PR50-mediated neurotoxicity but not from other DPRs (FIG. 17A), suggesting the rescue effect is specific to arginine-rich DPRs. This rescue was abolished by treating the neurons with the VPS34 inhibitor (FIG. 17B). Similar to neurons expressing poly-GR, the PR50-expressing neurons also exhibited deficits in PI3P levels, EEA1-positive early endosomes, and LAMP1-positive lysosomes, which were also diminished by PTPσ knockdown (FIGS. 17C-17E). PTPσ significantly modulates arginine-rich DPR (both GR and PR)-mediated neurotoxicity, through a similar PI3P-endosomal-lysosomal mechanism.

### Example 10

### ASOs Targeting PTPσ Improve Neuron Survival in Poly-GR/PR Expressing Neurons and C9-iMNs

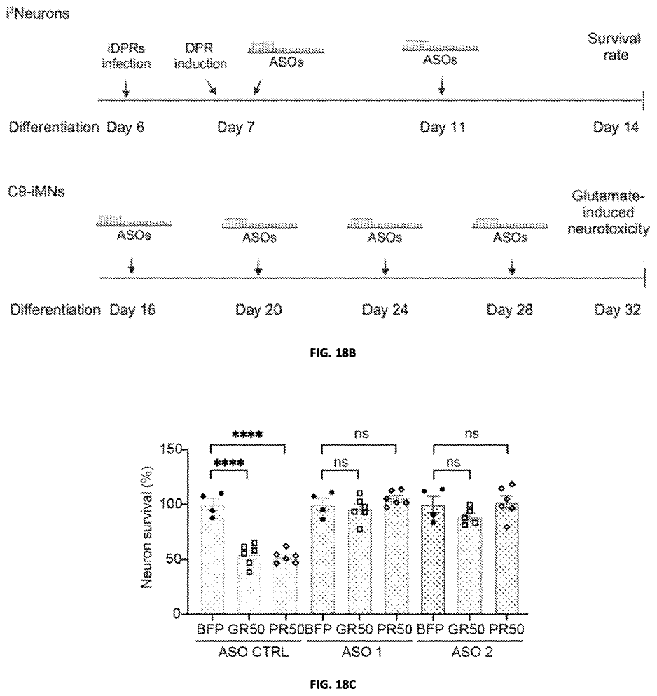

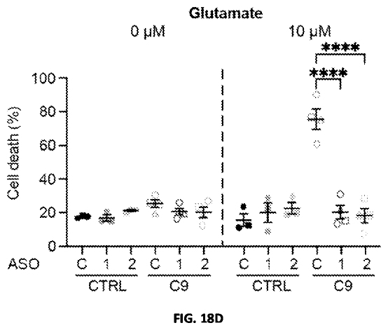

Antisense oligonucleotides (ASOs) have been increasingly employed as a therapeutic strategy in the neurogenerative diseases, including ALS. It binds to the target RNA and induces its degradation via RNase H1-mediated cleavage, thereby decreasing the expression level of the target gene. Observing the robust neuroprotective effect from PTPσ knockdown in poly-GR/PR expressing neurons and C9-iMNs, the potential of using PTPσ-targeting ASOs was explored. Two ASOs (ASO1: CAAGGACGCTTTCTACAGTG (SEQ ID NO: 20) and ASO2: TGACCTTACCCTCAGAACTG (SEQ ID NO: 21) modified with phosphorothioate bonds and 2′-O-methoxyethyl bases) were developed that efficiently reduced the PTPσ level (FIG. 18A). The ASO treatment (FIG. 18B) significantly enhanced the survival of GR50 or PR50 expressing neurons (FIG. 18C). Furthermore, the treatment of PTPσ ASOs on C9-iMNs (FIG. 18B) also significantly reduced the glutamate-induced neuro-excitotoxicity (FIG. 18D). Collectively, these results underscore the therapeutic potential of PTPσ-targeting ASOs for C9-ALS/FTD.

### Materials and Methods

Generation of plasmid. For Lenti-APEX2-GFP/BFP/GR50-FLAG: the PCR fragment containing HiBiT-APEX2-GFP/BFP/GR50-FLAG was digested with XbaI and SmaI and inserted into the XbaI and BamHI (blunt) sites of Lenti-UPF1-puro plasmid. For Lentiviral vectors expressing ATG14L: ATG14L was PCR amplified from pEGFP-Atg14L (Addgene 21635) and cut with AgeI and BsrGI, and inserted into the AgeI and BsrGI sites of Lenti-GFP-puro vector. For Lentiviral vectors expressing Rab5CA (Q79L), the GFP in the Lenti-GFP-puro construct was replaced with mCherry using AgeI and BsrGI. Rab5A (Q79L) was amplified from mCherry-Rab5A (Addgene, 35138) and subcloned in the vector via BsrGI and BamHI.

The Dox inducible lentiviral vector PLX304-TRE3G-MCS-Puro was generated as follows: the BSD fragment of lentivirus vector NLS-MCP-AP-PLX304 (Addgene #120918) was replaced by a Puro fragment with KpnI and EcoRI. Next, the PCR-amplified TRE3G promoters containing multiple cloning sites (MCS) replaced the Ubc-NLS-MCP-AP. The fragment encoding GA50, GP50, GR50, PA50 and PR50 with randomized codons were synthesized (Genewiz). The HiBiT tag with ATG start codon and 2× Flag was introduced to the N-terminus and C-terminus of those DNA fragments and inserted into PLX304-TRE3G-MCS-Puro.

Plasmids for AAV virus production (CAG-GR100-Flag): GR50 was expanded to GR100 using XhoI and SalI in pBluescript SK vector and subcloned to pcDNA5-FRT-TO HiBiT-HA-MCS-FLAG using XhoI and EcoRV. The HiBiT-HA-GR100-FLAG fragment was obtained by AflII and NotI digestion and cloned to pBluescript SK via SpeI and NotI. The HiBiT-HA-GR100-FLAG fragment was cut out by BamHI and NotI and cloned into the V506-pFB-CAG-full ITR vector (from Virovek) via BamHI and SalI.

For sgRNA, the pLG1 vector was cut with BstXI and BlpI and ligated with annealed oligos containing sgRNA sequences (PTPσ sgRNA F: TTGGGTCTCGGAGGCAGCCGAGTGTTTAAGAGC (SEQ ID NO: 2), PTPσ sgRNA R: TTAGCTCTTAAACACTCGGCTGCCTCCGAGACCCAACAAG (SEQ ID NO: 3)). For shRNA, the pLKO.1 non-targeting control shRNA plasmid (Addgene #10878) was cut with AgeI and EcoRI and ligated with annealed oligos containing shPTPσ or shTDP-43. The targeting sequences are as below: PTPσ shRNA (5′-CTGTATCCCGTGACATTTCAT-3′ (SEQ ID NO: 4), TDP-43 shRNA (5′-AAGCAAAGCCAAGATGAGCCT-3′ (SEQ ID NO: 5)). Other plasmids include: pLenti-EGFP-2×FYVE (Addgene, 136996), FUW mCherry-GFP LC3 (Addgene, 110060).

Lentivirus packaging and infection. Lentiviruses were produced as follows. HEK293 cells were transfected at 80-90% confluency with viral vectors containing genes of interest and viral packaging plasmids (PRRE, PREV and pVSVG) using Penefect™ plus transfection reagent (LifeSct). The medium was changed 16 h after transfection. Viruses were harvested at 72 h after transfection. Viral supernatants were collected and filtered with 0.45 μM filters. The supernatant was incubated with Lenti-X concentrator (Takara) in a ratio of 3:1 for at least 1 hour at 4° C. The mixture was centrifuged at 1,500 g at 4° C. for 45 min. The pellets were resuspended in 500 μl BP medium per 15 cm dish and stored at −80° C. AAV PHP.eB CAG-GR100-FLAG and CAG-GFP virus were packaged in Sf9 cells by Virovek at 2E10¹³ vg/mL.

iPSC maintenance and i³N differentiation. Human iPSCs engineered to express mNGN2 and dCas9 (male WTC11 background) were maintained in Essential 8 Medium (Gibco) on matrigel coated plates (StemCell Technologies) and passaged using acctuase when the confluency reached 80% as previous described (ref).

To start the differentiation, iPSCs were dissociated from the plate and resuspended in the N2 medium containing knockout DMEM/F12 (Gibco), 1×MEM Non-Essential Amino Acids (Gibco), 1×N2 Supplement (Gibco) and 2 mg/mL doxycycline (Sigma). 3 days after daily N2 medium change, N2 cells were seeded on PLO (Sigma) pre-treated plates in differentiation medium. The medium contains Brainphyis medium (StemCell Technology), 1×B27 (Gibco), 10 ng/mL NT-3 (PeproTech), 10 ng/mL BDNF (PeproTech), 1 mg/mL Mouse Laminin (Thermo Fisher Scientific), and 2 mg/mL doxycycline. Half of the media was exchanged every two days till harvesting on day 14.

C9 patient iPSNs differentiation (iMNs differentiation). Control or C9-iPSCs were grown in mTeSR medium (StemCell Technologies) on growth factor reduced matrigel coated plates (StemCell Technologies). Peripheral blood mononuclear cell (PBMC)-derived iPSC lines from C9ORF72-ALS patients, non-neurological disease controls were obtained from the Cedars-Sinai Answer ALS repository. iPSCs were grown in mTeSR plus medium (StemCell Technologies, Cat. No. 100-0276) on growth factor reduced Matrigel coated plates (StemCell Technologies, Cat. No. 354230). iPSCs were cultured in S1 medium containing IMDM (Gibco, Cat. No. 12440053), F12 (Gibco, Cat. No. 11765054), 1×NEAA, 1×B27, 1×N2, 1×PSA (Sigma, Cat. No. A5955), 0.2 μM LDN193189 (Sigma, Cat. No. SML0559), 10 μM SB431542 (StemCell Technologies, Cat. No. 72234) and 3 μM CHIR99021 (Cayman Chemical, Cat. No. 13122) to start the differentiation 5 days post passaging. After 6-day differentiation, cells were dissociated and passaged to S2 medium. S2 medium contains all the components of S1 medium with additional all trans retina acid (RA) at 0.1 μM (Sigma, Cat. No. R2625) and 1 μM SAG (Cayman Chemical, Cat. No. 11914). 6 days following passage (differentiation day 12), cells were seeded on Matrigel coated plates and cultured in S3 medium which contains IMDM, F12, NEAA, B27, N2, PSA, RA, SAG, 0.1 μM compound E (EMD-Millipore, Cat. No. 565790), 2.5 μM DAPT (Sigma, Cat. No. D5942), 0.1 μM db-cAMP (EMD-Millipore, Cat. No. 28745), 200 ng/ml ascorbic acid (Sigma, Cat. No. A4544), 10 ng/ml BDNF (Pepro Tech, Cat. No. 450-02) and 10 ng/mL GDNF (Pepro Tech, Cat. No. 450-10). On day 18, iPSNs (iMNs) were infected with control shRNA or PTPσ shRNA to knockdown PTPσ expression, followed by harvesting on day 32.

Cell survival assay. I'Neurons were infected with lentivirus to transduce GR50, PR50, BFP or GFP expression in the cells on day 5, day 7, day 9 or day 11 depending on the experimental timeline. For inducible DPRs, the virus was added to neurons on differentiation day 6. The expression was initiated by adding Dox to the differentiation medium (induction day 1). To validate the effect of the drugs on neuron survival, DPR was transduced for 7 days. i³Neurons were treated with 5 μM ASOs for 7 days or 2.5 μM ISP (Sigma, Cat. No. 5343390001) or 3.7 μM DJ001 (Sigma, Cat. No. SML2787) for 3 days before harvesting. To examine the effect of the drugs on other cellular phenotypes, DPR was transduced for 3 days. 2.5 μM ISP or 3.7 μM DJ001 was added to the culture medium on the same day, followed by FYVE reporter transduction 1 day later.

To measure cell survival, bright-field images were captured on day 14 using a Nikon TS-2 microscope. At least 4 images were taken at 10× object for each replica. At least 2 replicas were included for each batch. All experiments were performed at least twice. To examine dead neurons, PI (Thermo Fisher, Cat. No. P1304MP) dye was added to the culture medium 30 min before harvesting. Images of bright field and Cy3 channel were acquired, followed by quantifying the surviving neurons and PI⁺ signals blindly.

CRISPRi-Cas9 screening. The CRISPRi v2-h1 library (H1) contains sgRNAs targeting 2,318 genes encoding kinases, phosphatases, and drug targets (5 sgRNAs per gene), along with 250 non-targeting control sgRNAs (13,025 total elements). H1 library was packaged into lentivirus and infected into about 20 million iPSCs at a multiplicity of infection of 0.4. The infected cells were selected by puromycin treatment and further differentiated into neurons. To ensure the robustness of the screening, the cells were maintained at least 1,000-fold of the library complexity after drug selection and 500-fold at the end of the neuron stage. The i³N were infected on day 5 with either GR50 or GFP (non-toxic) by lentivirus and harvested on day 14. As the differentiation control, day-5 i³N were harvested as TO in duplicates. The genomic DNA was extracted using a NucleoSpin Blood L midi kit and libraries were constructed by PCR followed by gel purification for high-throughput sequencing on an Illumina NextSeq platform at a depth of at least 250 reads per element. Two replicas of each condition (TO, GFP and GR50) were analyzed by Cas9 high-throughput maximum-likelihood estimator (casTLE) algorithm to identify modifiers of GR50 toxicity. Briefly, guide compositions between GFP and GR50 to T0 were compared. The enrichment of individual sgRNA was calculated as the log ratios of different conditions, and gene-level effects were calculated from five sgRNAs targeting each gene.

Mouse studies. All procedures using mice were approved by Johns Hopkins University Animal Care and Use Committee (ACUC) (Protocol number M019M195). A balanced number of male and female animals were used for all the studies. Mice were randomly assigned to different treatment groups. Virus injection, drug injection, behavioral tests and cell quantification were performed by different individuals who are blind to the treatment. Mice were housed in the home cage with free access to food and water in a room with a 12 h light/12 h dark cycle.

Sample size estimation. Based on previous data, there is no significant difference between sex. 10-15 animals for each treatment yields robust results in the behavioral test. The sample size for the proposed study was determined by sample size calculator which is based on baseline incidence, population variance from the previous data and the probability of a type-1, type 2 error. 15 animals per group were determined to be necessary to evaluate the effect of the treatment in behavior.

Intracerebral ventricle (ICV) injection of virus in neonatal mouse brain. PO-1 neonates were immobilized via cryo-anesthesia for 2-3 minutes, then placed on a fiber-optic light to illuminate relevant anatomical structures that can be used as a guide. A micro-liter calibrated sterilized glass micropipette was attached to a 3 ml syringe, containing diluted 5×10⁹ AAV.PHP.eB virus (GFP or GR100-FLAG) in 2 μL PBS, through a long tube and inserted into the skull (2 mm penetration) at a location approximately 0.25 mm lateral to the sagittal suture and 0.50-0.75 mm rostral to the neonatal coronary suture. Following injection, pups were kept in a warmed container for 5-10 minutes and returned to their home-cage.

ISP administration in mice. AAV-GR100 was delivered to PO-1 C57B/6 mice and 5 μg of ISP in 100 μL PBS was injected daily starting from 1.5 months after virus delivery via subcutaneous injection until 3 months. The injection was stopped 3 days before the behavioral test to reduce the potential stress.

Behavioral tests. A set of behavioral tests were performed to assess the motor function and cognition in GFP and GR100 mice. Mice were housed in the home cage with free access to food and water in a room of 12 h light/12 h dark cycle. During the week of the behavioral tests, open field test was performed on day 1, followed by NOR on day 2-3 and rotarod on day 4. Mice were transferred to the facility to habituate for 1 h before the test and returned home cage after each test. Based on previous data, there is no significant difference between sexes in GR100 mice. 10-15 animals for each treatment yields robust results in the behavioral test. The sample size for the proposed study was determined by a sample size calculator based on baseline incidence, population variance from the previous data, and the probability of a type-1 or type-2 error. 15 animals per group were used to evaluate the effect of the treatment on behavior and at least 15 animals were included for the ISP administration trial.

Open field test. The white open-field arena had a diameter of 55 cm, and 55 cm high sidewalls. The same illumination was used, consisting of indirect diffuse room light. Each subject was released near the wall and observed for 20 min. Performance in the open field was recorded by a computer-based video tracking system (PAS). Activity measures included the grids break in the central and peripheral area of the apparatus and the rearing activity. Novel object recognition test. All observations were conducted in an open field (50 cm in diameter) with Plexiglas walls (40 cm in height). The objects were identical small cubes. A video camera was located directly over the center of the open field and was connected to a camera. Testing consisted of habitation and test phase with 5 min for each phase. For the habituation trial, two identical objects were placed into the open field (Object A and Object B). The test session was performed on the following day. On the test trial, one of the familiar objects (Object A) was replaced with a novel object (Object C) to evaluate recognition memory. In each trial, duration of contact with each object was recorded using a stopwatch. Discrimination index (DI)=(T object C−T object A)/(T object C+T object A).

Rotarod. For the rotarod test, each mouse was habituated to stay on the spindle with slow constant speed for 1 min, and then the spindle was accelerated from 4 r.p.m. to 40 r.p.m, increasing speed every 5 sec by 0.5 r.p.m. The latency to fall time was recorded when the mouse fell off the spindle.

Tissue harvesting and processing. Mice were perfused with DEPC-treated PBS. Sagittal half brain was fixed with 4% (v/v) paraformaldehyde, followed by sinking in 10%, 30% sucrose. 12 μm cryo-sections were serial sectioned from cryo-blocks to pre-treated glass slides for IF. The other half of the brain was snap frozen in liquid nitrogen for RNA, protein, and lip extraction.

Human tissues. Post-mortem temporal cortex and motor cortex tissues from C9ORF72 ALS/FTD patients, non-C9ORF72 FTD patients and control brain tissues were obtained from Johns Hopkins.

Immunofluorescence (IF) staining. For IF staining in cells, cells were fixed with 4% (v/v) paraformaldehyde in PBS for 10 min, permeabilized in 0.2% (v/v) Triton X-100 for 5 min, blocked in 1% bovine serum albumin and 2% goat serum for 1 hour. Cells were incubated with primary antibodies at 4° C. overnight. The primary antibody includes FLAG (Sigma, 1:200); poly-GR (Proteintech, 1:200); EEA1 (CST, 1:300); LAMP1 (CST, 1:200), MAP2 (D5G1, CST, 1:200); MAP2 (HM-2, Sigma, 1:300). On the second day, cells were washed with PBS, and incubated with Alexa Fluor 488/546/647 conjugated secondary antibodies (ThermoFisher Scientific). Nuclei were counterstained with Hochest 33342 or DAPI.

For IF staining in mice, cryo sections were brought to room temperature for at least 30 min before washing with PBS. After 3 times PBS wash, the sections were treated with in 0.2% (v/v) Triton X-100 for 10 min, blocked in 1% bovine serum albumin and 2% goat serum for 1 hour. Sections were incubated with primary antibodies at 4° C. overnight. The primary antibody includes poly-GR (Proteintech, 1:200); EEA1 (C45B10, CST, 1:300); NeuN (D3S3I, CST, 1:200), NeuN (A60, EMD Millipore, 1:100); GFAP (GA5, eBiosicence, 1:1000); Iba1 (Wako, 1:500). On the second day, sections were washed with PBS, and incubated with Alexa Fluor 488/546/647 conjugated secondary antibodies (ThermoFisher Scientific). Nuclei were counterstained with Hochest 33342 (ThermoFisher Scientific) or DAPI.

For IF staining in patient tissue, paraffin sections of human tissues were deparaffined, rehydrated through gradient ethanol to dH₂O. 100× Antigen unmasking solution (Tris-base, H-3301, Vector lab) was diluted with dH₂O. Slides were treated in the solution at 121° C., with high pressure for 20 min. After cooling down to room temperature, slides were treated with 0.2% Triton X-100 for 20 min, then blocked with 10% goat serum and 1% bovine serum albumin for 2 hours. Primary antibodies, EEA1 (C45B10, CST, 1:300) and NeuN (A60, EMD Millipore, 1:100) were diluted in the blocking containing 0.2% Triton X-100. Slides were incubated with primary at 4° C. overnight. The rest of the staining steps were the same as described above.

FITC-Dextran uptake assay. The assay was performed on day 14 for i³Neurons and day 23 for patient iPSNs seeded on coverslips. One day before the assay, a complete change of culture media was performed. A final concentration of 100 μg/mL FITC-Dextran (10 kDa) was added to the media. Sixteen hours after incubation, the media was exchanged with fresh media without Dextran. Two hours later, the coverslip was washed with PBS and then the cells were fixed with 4% PFA. The Nuclei were counterstained with Hochest 33342/DAPI 5 min before imaging. Images were taken at 63× using confocal microscopy.

RNA extraction, reverse transcription (RT-PCR) and quantitative PCR (qPCR). Trizol (Invitrogen) was used to isolate total RNA from cells. The first-strand cDNA was synthesized using the High-capacity cDNA reverse transcription kit following the manufacturer's instruction (Applied Biosystems). qPCR was performed with three biological replicates for each treatment using the LiQuant (Lifescience) on the CFX96 real-time PCR detection system (Bio-Rad). Expression values were normalized to Rp10 mRNA. Primer sequences are as below (5′-3′): TUBB3 F: AACGAGGCCTCTTCTCACAA (SEQ ID NO: 6), R: CCTCCGTGTAGTGACCCTTG (SEQ ID NO: 7); RBFOX3 F: CTTACGGAGCGGTCGTGTAG (SEQ ID NO: 8), R: AGAAGGAAACGGTGAAGGT (SEQ ID NO: 9); DLG4 F: TCGGTGACGACCCATCCAT (SEQ ID NO: 10), R: CGACGTCCACTTCATTTACAAAC (SEQ ID NO: 11); GPHN F: ACTAACCACGACCATCAAATCC (SEQ ID NO: 12), R: AGTCCCACCCAACAAAGAAGG (SEQ ID NO: 13); PTPσ F: GTCAAGGACTCGGCCAACTAC (SEQ ID NO: 14), R: GATTTCACCGTGATCTGAGCA (SEQ ID NO: 15); RPLPO F: TCTACAACCCTGAAGTGCTTGAT (SEQ ID NO: 16), R: CAATCTGCAGACAGACACTGG (SEQ ID NO: 17); GAPDHF: GAGTCAACGGATTTGGTCGT (SEQ ID NO: 18), R: TTGATTTTGGAGGGATCTCG (SEQ ID NO: 19).

Protein extraction and western blotting. Day 14 i³Neurons or Day 32 human iPSNs were rinsed with PBS and dissociated from the plate. Cell pellets were resuspended in RIPA buffer containing 1× protease inhibitor cocktail (Roche) and kept on ice for 20 min. Supernatant was harvested after 20 min centrifuge at 12,000 g. 20 μg of protein was mixed with 5× loading dye and boiled at 90° C. for 10 min. Samples were loaded onto a 10% SDS-PAGE gel. Wet transfer was performed to transfer the large sized protein (PTPσ). After 1 hour blocking with 5% skim milk, the bot was incubated with the primary antibody in 5% BSA at 4° C. overnight. Primary antibodies are PTPσ (Proteintech, Cat. No. 13008-1-AP, 1:1000), CC3 (CST, Cat. No. 96645, 1:1000), Rab5 (CST, Cat. No. 3547T, 1:1000), GAPDH (CST, Cat. No. 2118, 1:1000), B-actin (CST, Cat. No. 3700, 1:1000). On the second day, blots were washed with 1×TBST and probed with secondary antibody for 1 hour at room temperature. ECL was used to develop signal on the blot which was captured by BioRad imager.

Lipid extraction. 50 mg tissue from mouse cortex or human post-mortem brain was homogenized in trichloroacetic acid followed by lipid extraction using a non-radioactive method. Briefly, lipids were extracted using methanol and chloroform and then dried in a vacuum dryer. PI3P level was then measured by a competitive ELISA assay following the manufacturer's protocol (PI3P mass ELISA, Echelon Biosciences). Briefly, the dried lipid pellet was resuspended in PBST buffer (provided in the ELISA kit). Samples were incubated with PI3P detector protein and added to a PI3P-coated plate for competitive binding. A peroxidase-linked secondary detector was added to the plate, followed by colorimetric detection at 450 nm to detect PI3P binding.

PI3P ELISA. The dried lipid pellet was resuspended in PBST buffer (provided in the ELISA kit). Samples were incubated with PI3P detector protein and added to a PI3P coated plate for competitive binding. A peroxidase-linked secondary detector was then added to the plate, followed by colorimetric detection at 450 nm to detect PI3P binding to the plate.

Glutamate-induced excitotoxicity assay. To test the glutamate-induced excitotoxicity, control and C9-iMNs with control/PTPσ shRNA (14 days after infection), 5 μM ASOs (16 days after treatment), 2.5 μM ISP (3 days after treatment) or 3.7 μM DJ001 (3 days after treatment) were treated with 10 μM L-glutamate in artificial CSF for 4 hours. Then the cells were stained with Hoechst33342/DAPI (5 μg/ml) and Propidium Iodide (1 μg/ml) for 30 min to visualize total and dead cells, respectively. Five pairs of age and gender-matched control and C9ORF72-ALS iPSNs were analyzed.

Confocal microscopy and image quantification. Confocal images were captured using a Zeiss 980 Airyscan confocal microcopy. The laser power was kept consistent across all the replicas. Z stack and tile images were taken for iPSNs and mouse brains. For GFP-2×FYVE florescence intensity quantification, at least 6 images were taken for each replica with more than 30 neurons. Image J was used to analyze images. The contract was adjusted and then applied to all the images. The fluorescence intensity of the region of interest was measured and then minus the background intensity. For the quantification of EEA1⁺ and LAMP1⁺ puncta, EEA1 or LAMP1 channel was merged with MAP2. The puncta within MAP2 region were counted and compared among groups. For the quantification in mouse brains, at least 6 serial sections were imaged to quantify the number of neurons, astrocyte, and microglia in mouse brains. For glutamate-induced neurotoxicity assays, 5 images of PI staining will be taken at 20× for each well to quantify dead neurons. At least 500 cells from each biological replicate will be counted for statistical comparison.

Statistical analysis. Analysis was performed and graphed by Prism 9. Briefly, an F test was performed to examine the distribution of the data. For the groups that have equal SD (F test, P>0.05), a two-tailed student t-test was used for comparisons between the two groups. For multiple comparisons among groups, one-way ANOVA was used followed by Tukey's post hoc test. For comparisons of neuron survival under different conditions, two-way ANOVA with Tukey's post hoc test was used. For patient PI3P level of motor and temporal cortex comparison, two tailed student's t test was performed because of the huge variation human samples. For all tests, a p value inferior to 0.05 was taken as statistically significant.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

1. A method of treating a disease or disorder comprising administering to a subject in need thereof an effective amount of at least one protein tyrosine phosphatase sigma (PTPσ) inhibitor or a composition thereof, wherein the disease or disorder is characterized by the accumulation of dipeptide repeat (DPR) proteins.

2. The method of claim 1, wherein the dipeptide repeat (DPR) proteins comprise arginine-rich DPRs.

3. The method of claim 1, wherein the disease or disorder is a neurodegenerative disease or disorder.

4. The method of claim 1, wherein the disease or disorder is amyotrophic lateral sclerosis (ALS), frontotemporal dementia (FTD), or a combination thereof.

5. The method of claim 4, wherein the disease or disorder is C9ORF72-ALS/FTD.

6. The method of claim 1, wherein administration of the at least one PTPσ inhibitor reduces neuronal death, neuroinflammation, and progression of neurodegeneration.

7. The method of claim 1, wherein the PTPσ inhibitor is selected from the group consisting of proteins, nucleic acids, small molecules, and combinations thereof.

8. The method of claim 1, wherein the PTPσ inhibitor comprises an antisense oligonucleotide, a small interfering RNA (siRNA), a short hairpin RNA (shRNA), a guide RNA (gRNA) and combinations thereof.

9. The method of claim 1, wherein the PTPσ inhibitor comprises Intracellular Sigma Peptide (ISP), DJ001, or a combination thereof.

10. The method of claim 1, further comprising administering at least one additional therapeutic agent.

11. A method of enhancing survival, decreasing inflammation, and increasing endosome/lysosome function of a target cell comprising contacting a target cell with at least one protein tyrosine phosphatase sigma (PTPσ) inhibitor.

12. The method of claim 11, wherein the target cell is a neuron.

13. The method of claim 11, wherein the target cell has accumulation of dipeptide repeat (DPR) proteins.

14. The method of claim 13, wherein the dipeptide repeat (DPR) proteins comprise arginine-rich DPRs.

15. The method of claim 11, wherein contacting the target cell comprises providing the PTPσ inhibitor to an organ, tissue, cell, in vitro or ex vivo.

16. The method of claim 11, wherein contacting the target cell comprises administering the PTPσ inhibitor to a subject.

17. The method of claim 11, wherein the PTPσ inhibitor is selected from the group consisting of proteins, nucleic acids, small molecules, and combinations thereof.

18. The method of claim 11, wherein the PTPσ inhibitor comprises an antisense oligonucleotide, a small interfering RNA (siRNA), a short hairpin RNA (shRNA), a guide RNA (gRNA) and combinations thereof.

19. The method of claim 1, wherein the PTPσ inhibitor comprises Intracellular Sigma Peptide (ISP), DJ001, or a combination thereof.

20. (canceled)
